SEQUENCE LISTING (33 sequences)

SEQ ID NO: 1 - DNA, 668 bp, Escherichia coli
tgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctctggcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattaccgcgcagaaaaaaggatctcaagaagatcctttgatcttttctac

SEQ ID NO: 2 - DNA, 25 bp, Artificial
  AR ORIG ORI Primer (sense)
cactgattaagcattggtaactgtc

SEQ ID NO: 3 - DNA, 24 bp, Artificial
  AR Orig ORI Antisense Primer
aggcacctatctcagcgatctgtc

SEQ ID NO: 4 - DNA, 31 bp, Artificial
  DNA Long Primer (sense)
cactgattaagcattggtaactgtctatttc

SEQ ID NO: 5 - DNA, 29 bp, Artificial
  DNA long Antisense Primer
aggcacctatctcagcgatctgtcagacc

SEQ ID NO: 6 - DNA, 25 bp, Artificial
  LNA-9 version Primer (sense)
cactgattaagcattggtaactgtc

SEQ ID NO: 7 - DNA, 24 bp, Artificial
  LNA-9 version Antisense Primer
aggcacctatctcagcgatctgtc

SEQ ID NO: 8 - DNA, 20 bp, Artificial
  UniOri 20 Primer (sense)
ctgaagccagttaccttcgg

SEQ ID NO: 9 - DNA, 19 bp, Artificial
  UniOri20 Antisense Primer
cagagctcagataccaaat

SEQ ID NO: 10 - DNA, 22 bp, Artificial
  UniOri 22 Primer (sense)
ctgaagccagttaccttcggaa

SEQ ID NO: 11 - DNA, 22 bp, Artificial
  UniOri22 Antisense Primer
cagagcgcagataccaaatact

SEQ ID NO: 12 - DNA, 25 bp, Artificial
  UniOri25 Primer (sense)
ctgaagccagttaccttcggaaaaa

SEQ ID NO: 13 - DNA, 25 bp, Artificial
  UniOri25 Antisense Primer
cagagcgcagataccaaatactgtc

SEQ ID NO: 14 - DNA, 30 bp, Artificial
  UniOri30 Primer (sense)
ctgaagccagttaccttcggaaaaagagtt

SEQ ID NO: 15 - DNA, 30 bp, Artificial
  UniOri30 Antisense Primer
cagagcgcagataccaaatactgtccttct

SEQ ID NO: 16 - DNA, 20 bp, Artificial
  20mer Sense
ctgaagccagttaccttcgg

SEQ ID NO: 17 - DNA, 19 bp, Artificial
  20mer Antisense
cagagcgcagataccaaat

SEQ ID NO: 18 - DNA, 22 bp, Artificial
  22mer Antisense
cagagcgcagataccaaatact

SEQ ID NO: 19 - DNA, 22 bp, Artificial
  22mer Sense
ctgaagccagttaccttcggaa

SEQ ID NO: 20 - DNA, 25 bp, Artificial
  25mers Sense
ctgaagccagttaccttcggaaaaa

SEQ ID NO: 21 - DNA, 25 bp, Artificial
  25mers (antisense)
cagagcgcagataccaaatactgtc

SEQ ID NO: 22 - DNA, 30 bp, Artificial; identical to SEQ ID NO: 14
  30mers Sense

SEQ ID NO: 23 - DNA, 30 bp, Artificial; identical to SEQ ID NO: 15
  30mer Primer (antisense)

SEQ ID NO: 24 - DNA, 25 bp, Artificial
  LNA/phosphorothioate Primer Set A - Sense
cactgattaagcattggtaactgtc

SEQ ID NO: 25 - DNA, 24 bp, Artificial
  LNA/phosphorothioate Primer Set A - Antisense
aggcacctatctcagcgatctgtc

SEQ ID NO: 26 - DNA, 25 bp, Artificial
  LNA/phosphorothioate Primer Set B - Sense
cactgattaagcattggtaactgtc

SEQ ID NO: 27 - DNA, 24 bp, Artificial
  LNA/phosphorothiate Primer Set B - Antisense
aggcacctatctcagcgatctgtc

SEQ ID NO: 28 - DNA, 25 bp, Artificial
  LNA/phosphorothioate Primer Set C - Sense
cactgattaagcattggtaactgtc

SEQ ID NO: 29 - DNA, 24 bp, Artificial
  LNA/phosphorothioate Primer Set C - Antisense
aggcacctatctcagcgatctgtc

SEQ ID NO: 30 - DNA, 25 bp, Artificial
  LNA/phosphorothioate Primer Set D - Sense
cactgattaagcattggtaactgtc

SEQ ID NO: 31 - DNA, 24 bp, Artificial
  LNA/phosphorothioate Primer Set D - Antisense
aggcacctatctcagcgatctgtc

SEQ ID NO: 32 - DNA, 29 bp, Artificial
  LNA/phosphorothioate Primer Set E - Sense
cnancntngattaagcattggtaactgtc

SEQ ID NO: 33 - DNA, 28 bp, Artificial
  LNA/phosphorothioate Primer Set D - Antisense
angngncnacctatctcagcgatctgtc

(19) United States
(12) Patent Application Publication
(10) Pub. No.: US 2005/0244847 A1
(43) Pub. Date: Nov. 3, 2005

(54) Methods and compositions for in vitro amplification of extrachromosomal nucleic acid

(75) Inventors: Michael J. Domanico, Middleton, WI (US); Lars-Erik Peters, Lafayette, CO (US); Preston Hradecky, Longmont, CO (US); Allan Roberts, Thornton, CO (US); David Latorra, Boulder, CO (US); Elaine Best, Fort Collins, CO (US)

Correspondence Address: DORSEY & WHITNEY, LLP;INTELLECTUAL PROPERTY DEPARTMENT, 370 SEVENTEENTH STREET, SUITE 4700, DENVER, CO, 80202-5647, US

(73) Assignee: Eppendorf AG

(21) Appl. No.: 11/011,527

(22) Filed: Nov. 26, 2004

Related U.S. Application Data
(60) Provisional application No. 60/525,459, filed on Nov. 26, 2003.

(51) Int. Cl. C12Q 1/68; C12P 19/34

(52) U.S. Cl. 435/6; 435/91.2

(57) ABSTRACT

Compositions and methods are provided for the in vitro amplification of nucleic acid and, in particular, for the amplification of extrachromosomal nucleic acid molecules having a molecular weight of one kilobase or greater. The design and use of primers, low ionic strength amplification buffer, low co-solvent containing buffer, and combined polymerization/ligation reaction buffers are described in this regard.

### CROSS-REFERENCE TO RELATED APPLICATIONS

The instant application claims priority to U.S. Provisional Application Ser. No. 60/525,459, filed on Nov. 26, 2003, entitled METHODS AND COMPOSITIONS FOR IN VITRO AMPLIFICATION OF EXTRACHROMOSOMAL NUCLEIC ACID, which is herein incorporated by reference.

### FIELD OF THE INVENTION

The invention generally relates to methods and compositions for the in vitro amplification of nucleic acid molecules and more particularly to the in vitro amplification of plasmids and other like nucleic acid.

### BACKGROUND OF THE INVENTION

The ability to prepare substantial amounts of nucleic acid molecules, especially nucleic acid molecules larger than one kilobase in size, for example extrachromosomal DNA such as, e.g., plasmid DNA, is requisite to a number of protocols in molecular biology, as well as a basic requirement in many downstream uses in biotechnology and clinical research. For example, larger molecular weight nucleic acid molecules are often preferred in cloning experiments, DNA sequencing reactions, restriction digestion reactions, and subsequent ligation reactions, and these uses are all, or to some extent, dependent on quality and quantity of the starting DNA material. As such, there has been, and continues to be, a need for reliable methods for producing large amounts of quality nucleic acid molecules, especially with respect to extrachromosomal DNA, and plasmid DNA in particular.

The predominant approach for preparing large amounts of nucleic acid is via the polymerase chain reaction (PCR). PCR is a convenient in vitro amplification method useful in the exponential increase of template nucleic acid. However, PCR has traditionally been used for amplification of lower molecular weight nucleic acid targets, e.g., less than 2 kb, and has typically resulted in unsatisfactory results (both quality and quantity) in the amplification of nucleic acid molecules larger than 1 kb in size. In general, the failure to amplify satisfactory levels and/or quality of 1 plus kb nucleic acid molecules stems from the polymerases' inability to accurately amplify that number of nucleotides, and/or to remain on longer templates for the period of time required to amplify the entire length of the product. Several approaches have been taken to modify PCR for amplification of these larger molecular weight molecules, including modifying PCR buffering capacity, for example inclusion of tricine-NH₄ or tricine-KOH, into the reaction mix, inclusion of PCR enhancers like DMSO into the reaction mix, and modifications of the polymerase enzyme itself. With regard to modifications in the PCR buffer, the inclusions result in a significant impact on the ionic strength of the buffer and the fidelity of the polymerase itself, and thus any resulting increase in amplification often comes at the expense of fidelity. With respect to modifications on the polymerase enzyme itself, some level of improvement has been obtained in fidelity and processivity, but at a cost of incorporating expensive hybrid proteins that often don't justify the change.

More recently, new amplification techniques have been developed to amplify closed circular nucleic acid molecules, especially in the 1-2 kb range, although amplification of a 10 kb plasmid has also been described. In particular, a single “amplification” reaction, termed a ligation during amplification (LDA) reaction, was developed to include a mixture of a thermostable polymerase, a thermostable ligase, and an energy source (See U.S. Pat. No. 6,620,597). Based on the data presented in the patent, it is unclear whether larger nucleic acid molecules in the 10 to 20 kb range can be successfully amplified with this technique, and more significantly, there is no demonstration that the fidelity of the amplified target, including smaller targets, is acceptable for any application of nucleic acid molecules given the large number of amplified bases. Since LDA combines polymerization with ligation buffering conditions, it is anticipated that ligation will not be efficient.

Consequently, a number of extrachromosomal nucleic acid molecules, e.g., plasmids, BACs, cosmids, YACs, and the like, having appropriate replicative signals, are still generally prepared by culturing host cells and subsequently purifying the target molecules using well known methods in the art, e.g., miniprep or maxiprep-derived protocols. In particular, target host cells are transformed with the extrachromosomal nucleic acid molecule, placed under selective pressure to ensure that the host cell harbors and maintains the extrachromosomal nucleic acid, and the host cells are then grown to levels sufficient to provide the anticipated amount of required extrachromosomal nucleic acid. Once grown, host cells are lysed and the contents solubilized. The target extrachromosomal nucleic acid is then separated from the contaminating host cell protein, RNA, genomic DNA, and other macromolecules via one of several commonly used chemical or enzymatic methods.

In general, the overall process of preparing larger amounts of extrachromosomal nucleic acid is fairly labor intensive, time consuming, and requiring of specialized laboratory equipment, i.e., host cell growth platforms, equipment necessary to sterilize host cell growth media (or the wherewithal to purchase the media), larger rotor centrifuges, autoclaves, pyrex containers, sterile tubes, etc. As such, preparation of extrachromosomal nucleic acid molecules is often limited to those laboratories having sufficient time and equipment to manipulate host cells, or those that can afford to have the cells grown and maintained for them. In addition, these culture and purification methods are not adaptable to target nucleic acid molecules that do not replicate in a host cell or with host cells that are not culturable. Furthermore, extrachromosomal nucleic acid prepared in this manner is often isolated along with fairly high levels of endotoxin. As such, extrachromosomal nucleic acid isolated via miniprep/maxiprep methods is often unsatisfactory for follow-up uses that require the use of endotoxin sensitive enzymes.

In addition, conventional culturing and purification methods of extrachromosomal nucleic acid can vary dependent upon a number of interconnected and complex factors, including: the size (base pairs) and number (copy number per cell) of extrachromosomal nucleic acid molecules harbored within each host cell, the type/strain of host cell used to prepare the nucleic acid, the growth rate of the host cell, and the amount of time the host cells are allowed to grow before isolation of the extrachromosomal nucleic acid from the host cell. For example, preparation of a low copy extrachromosomal nucleic acid will yield significantly lower amounts than the levels of high copy extrachromosomal nucleic acid molecules under the same growth and harvest conditions. As such, conventional methods used to produce extrachromosomal nucleic acid can yield inconsistent amounts of product. Taken together, conventional methods for extrachromosomal nucleic acid preparation provide room for improvement in both ease and affordability of the procedure as well as the effectiveness and reliability of the result.

As such, there is a continuing need in the art for a simple, consistent and time efficient method for preparing nucleic acid molecules, especially nucleic acid molecules greater than 1 kb in size, including extrachromosomal nucleic acid molecules, as well as for improved reagents and primers capable of use therein. In particular, there is a need for methods and reagents for preparing sufficient amounts of extrachromosomal nucleic acid that eliminate the time, expense and inconsistency of existing conventional methods as well as limitations associated with PCR in these larger molecular weight nucleic acid molecules, for example, on most extrachromosomal elements.

Against this backdrop the present invention has been developed.

### SUMMARY OF INVENTION

The present invention provides compositions and methods for the in vitro amplification of extrachromosomal nucleic acid, and in particular, for the in vitro amplification of extrachromosomal nucleic acid having a molecular weight of at least about 1 kb, and more preferably about 2-30 kb such as, e.g., plasmid DNA. Amplification methods in accordance with the present invention can be performed on linear or circular single- or double-stranded template DNA, and amplification products, i.e., amplicons, are generally linear. In preferred embodiments, the methods utilize double-stranded circular template DNA.

As demonstrated herein, the fidelity of the amplicons resulting from the methods and compositions provided herein is excellent, and the resulting preparations have substantially reduced endotoxin levels. Linear products formed using the subject methods can be utilized as linear products or further ligated into closed, circular, nucleic acid molecules using a standard ligation reaction or utilizing host cell in vivo ligation processes. Alternatively, reaction conditions are provided for the substantially simultaneous amplification and ligation of nucleic acid molecules into closed circular products within the same reaction buffer. Further, linear products can be targeted as a template for additional amplification reactions.

In one aspect, methods for amplifying a circular nucleic acid template are provided, comprising contacting the template with a reaction mixture comprising a thermostable polymerase, individual nucleotides and forward and reverse primers complementary to a common region within the template, wherein the common region is preferably from about 80 to 150 base pairs in length. In one embodiment, the 5′ ends of the primers hybridize to opposite strands of the template about 10 to 50 base pairs apart, still more preferably from zero to twenty-five base pairs apart. As described herein, the 5′ end of the forward primer will generally be proximal to the 5′ end of the reverse primer and distal to the 3′ end of the reverse primer when the primers are hybridized to the template. In a particularly preferred embodiment, the common region is a conserved region, e.g., an origin of replication, within an extrachromosomal nucleic acid. The reaction mixture may further include a reaction buffer comprising a weak organic base and a weak organic acid.

The invention also provides improved reagents for performing in vitro amplification of extrachromosomal DNA, including solutions supporting amplification and subsequent ligation reactions and solutions supporting a combined amplification and ligation reaction, i.e., which provide the appropriate environment for simultaneous polymerase and ligase enzyme activity.

In one aspect, compositions in accordance with the present invention comprise a buffering system having significant buffering capacity that supports high fidelity as well as highly processive amplification. In one embodiment, a composition for amplifying episomal genetic elements includes a thermostable polymerase, a weak organic base, a weak organic acid, a magnesium-containing compound, and a salt having a concentration of about 3 mM to 300 mM. Typically, the pH of the composition is modified by the combination of the weak organic base and weak organic acid to be between about pH 7.9 to about pH 8.9, and preferably from between a pH of 8.8 and 8.9. In a further embodiment, the buffering system of the invention provides enhanced Mg²⁺ concentration buffering capacity during the amplification reaction.

In another aspect, the invention provides forward and reverse primer pairs for use in the subject methods having 5′ ends that hybridize in close proximity with each other on opposite strands of a double-stranded circular nucleic acid template, preferably within about 50 base pairs, more preferably within about 10 base pairs, still more preferably within about 0 to 5 base pairs of each other. In preferred embodiments, the 5′ end of the forward primer is proximal to the 5′ end of the reverse primer and distal to the 3′ end of the reverse primer around the circumference of the circular template when the primers are hybridized to their respective strands of the template. In a particularly preferred embodiment, forward and reverse primers are provided that hybridize to adjacent base pairs on the sense and antisense strands of the template, respectively.

In a further aspect, universal primers are provided for use in amplifying a particular group of episomal genetic elements, having sequences complementary to a portion of a conserved region of the desired group, for example, an origin of replication of a bacterial plasmid. Conserved regions contemplated for use in the subject compositions and methods include, e.g., a bacterial origin of replication, a yeast two micron origin, drug resistance genes, and promoter sequences. Both the position and orientation of novel primers are provided for a number of exemplary primer pairs for different extrachromosomal nucleic acid molecules.

In another aspect, methods for preparing multiple copies of extrachromosomal DNA, for example, plasmid DNA, having lower endotoxin levels are provided. The subject methods comprise harvesting from one to twenty, more preferably from one to ten, still more preferably from one to five, and most preferably from one to three colonies of cells harboring a plasmid of interest and contacting the harvested cells with an amplification and optionally a ligation composition, the composition comprising: forward and reverse primers having sequences complementary to opposite strands within a conserved region of the plasmid; a buffer having a weak organic acid and weak organic base with a pH of between about 7.9 and about 8.9; a thermostable polymerase; optionally a thermostable ligase; and thermally cycling the composition for a plurality of cycles through a denaturation temperature, an annealing temperature, an elongation temperature and optionally a ligation temperature wherein a portion of the amplified DNA is closed circular plasmid DNA.

In a further aspect, methods for the in vitro amplification of closed circular plasmid DNA from a colony, the plasmid having an origin of replication, are provided. The method comprises contacting plasmid DNA from harvested cells with a composition comprising: a thermostable polymerase, individual nucleotides and forward and reverse primers complementary to the origin of replication of the plasmid DNA, wherein the forward primer hybridizes to the sense strand of the plasmid and the reverse primer hybridizes to the antisense strand of the plasmid within the origin of replication; and subjecting the resulting reaction mixture to thermal cycling to facilitate the polymerization of new linear copies of the plasmid DNA. Optionally, as further described herein, the newly formed linear copies may be ligated into closed circular plasmid DNA simultaneously or sequentially with the amplification step.

These and various other features and advantages of the invention will be apparent from a reading of the following detailed description and a review of the appended claims.

### BRIEF DESCRIPTION OF THE DRAWINGS

FIGS. 1A-D are flow diagrams illustrating several primer orientation designs: (1A) tail-to-tail; (1B) overhanging; (1C) primer tails incorporating a restriction site; and (1D) primers having a gap configuration in accordance with the present invention.

FIG. 2 is a stained agarose gel showing amplicons formed using an embodiment of the amplification buffer and novel primer pairs of varying length in accordance with the present invention. Several different episomal genetic elements were amplified using one of six different universal primer pairs.

FIG. 3 is a stained agarose gel showing amplicons formed using an embodiment of the amplification buffer and novel primer pairs for several different episomal genetic elements. Amplification reactions were performed directly on re-suspended colony picked cells.

FIG. 4 is a stained agarose gel showing amplicons formed using an embodiment of the amplification buffer and novel primer pairs from several template sources—purified plasmid DNA, re-suspended colony DNA and glycerol stock DNA.

FIG. 5 is a stained agarose gel showing the yield of amplicons formed using an embodiment of the amplification buffer and novel primer pairs from several sources. DNA was sequenced using ABI standard conditions to illustrate the quality of the amplified DNA.

FIGS. 6A and 6B show stained agarose gels that illustrate the effectiveness of a one-step amplification and ligation reaction directly on colony picked plasmid DNA. FIG. 6A shows a polymerase titration in a standard concentration of Tsc DNA ligase for a pUC19 target using the compositions and methods of the invention and FIG. 6B shows a Tsc DNA ligase titration in a standard concentration of polymerase (TripleMaster®) using the compositions and methods of the invention.

FIG. 7 is a stained agarose gel that illustrates the effectiveness of preparing a one-step amplification and ligation reaction directly on pUC19 cells. These results indicate in the presence of Tsc DNA ligase a covalently closed circular plasmid is formed. Also demonstrated in this experiment is the resistance to Dpn I digestion by vector amplified DNA. Purified plasmid DNA is methylated and therefore cleaved by Dpn I whereas vector-amplified DNA is non-methylated.

FIG. 8 is a stained agarose gel showing the yield of amplicons formed using an embodiment of the amplification buffer and novel primer pairs in the presence of increasing amounts of Mg²⁺.

FIGS. 9A and 9B are stained agarose gels showing temperature dependence of the annealing step of the amplification reaction on different length primers (A) and optimization of the two-step PCR in accordance with embodiments of the present invention.

FIG. 10 is a stained agarose gel showing DNA amplified directly from cells using methods and compositions in accordance with the present invention.

FIG. 11 is a stained gel showing DNA amplified using LNA modified primers in accordance with embodiments of the present invention.

### DETAILED DESCRIPTION OF THE INVENTION

Definitions:

The following definitions are provided to facilitate understanding of certain terms used frequently herein and are not meant to limit the scope of the present disclosure:

As used herein, “antisense” refers to polynucleotide sequences that are complementary to target “sense” polynucleotide sequences.

As used herein, “bicine” or “N,N-Bis(2-hydroxyethyl)glycine” refers to a compound having the general formula C₆H₁₃NO₄ and includes derivatives and salts of the compound. Bicine can be purchased from Hampton Research, Aliso Viejo, Calif., Product No. HR2-509.

As used herein, “extrachromosomal nucleic acid” refers to certain integrated and non-integrated episomal genetic elements, e.g., certain DNA viruses as well as natural plasmids, plasmid-derived cloning and expression vectors, natural bacterial phages and cloning and expression phages, yeast artificial chromosomes (YAC), bacterial artificial chromosomes (BAC), two micron DNA, two-micron derived yeast shuttle vectors, mitochondrial DNA, cosmids, phagemids, transposons, and the like.

As used herein, “nucleic acid” or “NA” refers to both a deoxyribonucleic acid (DNA) and a ribonucleic acid (RNA), as well as modified and/or functionalized versions thereof. Similarly, the term “nucleotide” as used herein includes both individual units of ribonucleic acid and deoxyribonucleic acid as well as nucleoside and nucleotide analogs, and modified nucleotides such as labeled nucleotides. In addition, “nucleotide” includes non-naturally occurring analog structures, such as those in which the sugar, phosphate, and/or base units are absent or replaced by other chemical structures. Thus, the term “nucleotide” encompasses individual peptide nucleic acid (PNA) (Nielsen et al., Bioconjug. Chem. 1994; 5(1):3-7) and locked nucleic acid (LNA) (Braasch and Corey, Chem. Biol. 2001; 8(1):1-7) units as well as other like units.

As used herein, “polynucleotide,” “oligonucleotide” or grammatical equivalents thereof means at least two nucleotides covalently linked together. As will be appreciated by those of skill in the art, various modifications of the sugar-phosphate backbone may be done to increase the stability of such molecules in physiological environments, including chemical modification such as, e.g., phosphorothioate or methyl phosphonate. Further, such molecules may be functionalized by coupling with one or more molecules having distinct characteristic properties for purposes of, e.g., facilitating the addition of labels.

As used herein, “nucleic acid sequence” refers to the order or sequence of nucleotides along a strand of nucleic acids. In some cases, the order of these nucleotides may determine the order of the amino acids along a corresponding polypeptide chain. The nucleotide sequence thus codes for the amino acid sequence. The nucleic acid sequence may be single-stranded or double-stranded, as specified, or contain portions of both double-stranded and single-stranded sequences. The nucleic acid sequence may be composed of DNA, both genomic and cDNA, RNA, or a hybrid, where the sequence comprises any combination of deoxyribo- and ribo-nucleotides, and any combination of bases, including uracil (U), adenine (A), thymine (T), cytosine (C), guanine (G), inosine, xathanine hypoxathanine, isocytosine, isoguanine, etc.

As used herein, “circular nucleic acid template” or “circular template” refers to circularized nucleic acid molecules, and preferably to heterologous, extrachromosomal circularized nucleic acid sequences such as vectors and expression cassettes and the like, typically having a molecular weight of approximately 2 kb to 45 kb, and in some aspects from 2 kb to 25 kb, and in other aspects from 2 kb to 10 kb. In preferred embodiments, the circular nucleic acid of the invention comprises a plasmid, where the plasmid comprises an origin of replication or replicator, a selectable marker and a cloning site. In some instances the circular nucleic acid is supercoiled. The circular nucleic acid template can include most episomal genetic elements, e.g., plasmids, cosmids, PACs, BACs, YACs, fosmids, and other like molecules.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. As is well known in the art, the degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method, as is well known in the art.

A primer is selected to be complementary to a strand of specific sequence of the template. A primer must be sufficiently complementary to hybridize with a template strand for primer elongation to occur. A primer sequence need not reflect the exact sequence of the template. For example, as disclosed herein, a non-complementary polynucleotide fragment may be attached to the 5′ end of the primer, with the remainder of the primer sequence being substantially complementary to the strand. Non-complementary bases or longer sequences can be interspersed into the primer, provided that the primer sequence has sufficient complementarity with the sequence of the template to specifically hybridize and thereby form a template primer complex for synthesis of the extension product of the primer at a predetermined temperature. Conditions of hybridization are standard as described in Sambrook et al., Eds., “Molecular Cloning, A Laboratory Manual” 3^{rd} Edition (Cold Spring Harbor Laboratory Press 2001) and discussed in more detail herein.

As used herein, “expression” refers to transcription and translation occurring within a host cell. The level of expression of a DNA molecule in a host cell may be determined on the basis of either the amount of corresponding mRNA that is present within the cell or the amount of the DNA-encoded protein produced by the host cells. Further detail for the term “expression” within the context of the present invention can be obtained via a review of Sambrook et al., supra.

As used herein, “host cell” or “host cells” refers to cells expressing or capable of expressing a heterologous polynucleotide molecule, for example a plasmid vector. Host cells of the present invention express polynucleotides encoding polypeptides having any number of uses, including biotechnological, molecular biological and clinical settings. Examples of suitable host cells in the present invention include, but are not limited to, bacterial, yeast, insect and mammalian cells. Specific examples of such cells include, E. Coli DH5α cells, as well as various other bacterial cell sources, for example the E. Coli strains: DH10b cells, XL1Blue cells, XL2Blue cells, Top10 cells, HB101 cells, and DH12S cells, and yeast host cells from the genera including Saccharomyces, Pichia, and Kluveromyces.

As used herein, “isolated” and “purified” for purposes of the present invention are interchangeable, and refer to a polynucleotide or polypeptide, for example extrachromosomal nucleic acid, that has been separated from cellular debris, for example, high molecular weight DNA, RNA and other protein. This would include an isolated RNA sample that would be separated from cellular debris, including DNA.

As used herein, “PHRED” or “PHRED score” refers to a software program used to measure DNA sequence quality. The software is purchased from CodonCode Corporation, version 0.020425.c. For purposes of the present invention, a PHRED q20 score of 600 is equivalent to approximately 730 bases at >98.5% accuracy.

As used herein, “plasmid” refers to a non-chromosomal, circular strand of nucleic acid found in certain types of bacteria. Preferably, circular double stranded nucleic acid molecules. Exemplary plasmids useful in the invention include pGEM, pTZ, pUC19, pUC18, pBS2, pEGFP, pBR322, and the like.

As used herein, “primer” refers to a single-stranded oligonucleotide which acts as a point of initiation of template-driven NA synthesis under appropriate conditions, i.e., compositions, buffers and temperatures. Length of a primer in accordance with the present invention is discussed in more detail below. A primer need not reflect the exact sequence of the template, for example a portion of the origin of replication, but must be sufficiently complementary to hybridize with a template. A forward primer is one that hybridizes in a 5′ to 3′ direction along a double-stranded template and a reverse primer is one that hybridizes to the complementary strand in a 3′ to 5′ direction relative to the first strand.

As used herein, “selectable marker” refers to a gene on extrachromosomal DNA typically used to select a target extrachromosomal DNA. Selectable markers include, for example, genes that encode antimetabolite resistance such as the DHFR protein that confers resistance to methotrexate (Wigler et al., 1980, Proc Natl Acad Sci USA, 77:3567; O'Hare et al., 1981, Proc Natl Acad Sci USA, 78:1527), the GPT protein that confers resistance to mycophenolic acid (Mulligan & Berg, 1981, PNAS USA, 78:2072), antibiotic resistance, i.e., neomycin resistance marker that confers resistance to the aminoglycoside G-418 (Calberre-Garapin et al., 1981, J Mol Biol, 150:1).

As used herein, “stringency” refers to the conditions, i.e., temperature, ionic strength, solvents, and the like, under which hybridization between polynucleotides occurs. Hybridization being the process that occurs between the primer and template DNA during the annealing step of the amplification process.

As used herein, “vector,” “extra-chromosomal vector” or “expression vector” refers to a circular polynucleotide molecule, usually double-stranded, which may have a site for insertion or have inserted a target heterologous polynucleotide. The heterologous polynucleotide molecule may or may not be naturally found in the host cell, and may be, for example, one or more additional copy of the heterologous polynucleotide naturally present in the host genome. The vector is adapted for transporting the foreign polynucleotide molecule into a suitable host cell. Once in the host cell, the vector may be capable of integrating into the host chromosomes. The vector may optionally contain additional elements for selecting cells containing the integrated polynucleotide molecule as well as elements to promote transcription of MRNA from transfected DNA. Examples of vectors useful in the methods of the present invention, include, but are not limited to, plasmids, bacteriophages, cosmids, retroviruses, and artificial chromosomes.

As used herein, “weak organic acid” refers to and includes, but is not limited to, bicine, tricine, TAPSO, CAPSO, EPPS, Hepes, CHES, Taurin, MOPS, AMPSO and the like.

As used herein, “weak organic base” refers to and includes, but is not limited to, tris, bis-tris, imidazole, and bis-tris-propane. In general weak organic bases have a pK between 8 and 9 for purposes of the present invention, although slight modifications above 8 and 9 are envisioned to be within the scope to the disclosure.

“Proximal” and “distal” are used herein in relation to each other. That is, where A is referred to as proximal to B and distal to C, the distance from A to B is less than the distance from A to C.

The compositions and methods provided herein find advantageous use in the amplification of nucleic acids, and in the amplification of circular nucleic acid templates such as extrachromosomal nucleic acids in particular. As is well known in the art, amplification reactions based on the polymerase chain reaction generally involve 1) denaturation of a double-stranded nucleic acid template at a high temperature; 2) annealing of primers to the template nucleic acid at a lower temperature; and 3) extension of the primers using a DNA polymerase, and preferably a thermostable DNA polymerase. See, e.g., U.S. Pat. Nos. 4,683,195; 4,683,202; and 4,965,188, each of which is hereby incorporated by reference. As described herein, the annealing phase and the extension phase may occur at the same or different temperatures, and are preferably accomplished at the same temperature. The subject invention provides primers and buffers which enhance these methods, resulting in amplicons having high fidelity for use in subsequent sequencing, cloning and detection assays.

Primer Selection

Primer design and selection of the invention includes primers for amplifying an entire extrachromosomal nucleic acid, for example, a plasmid or cosmid. Primer pairs of the invention are designed to anneal on opposite strands of a double-stranded template as is well known in the art. However, unlike traditional PCR primer pairs where each primer is designed to bracket a target insert and the 3′ ends of each primer are proximal to each other, primers of the present invention are designed to anneal on opposite strands of the template target such that the 5′ end of the forward (i.e. sense) primer is proximal to the 5′ end of the reverse (i.e. antisense) primer and distal to the 3′ end of the reverse primer. Hence, the 5′ ends of the primers preferably hybridize within the same 150 base pair region, more preferably within about 80-100 base pairs of each other, still more preferably within about 25-50 base pairs, and most preferably within close proximity of each other, e.g., 25 base pairs or less.

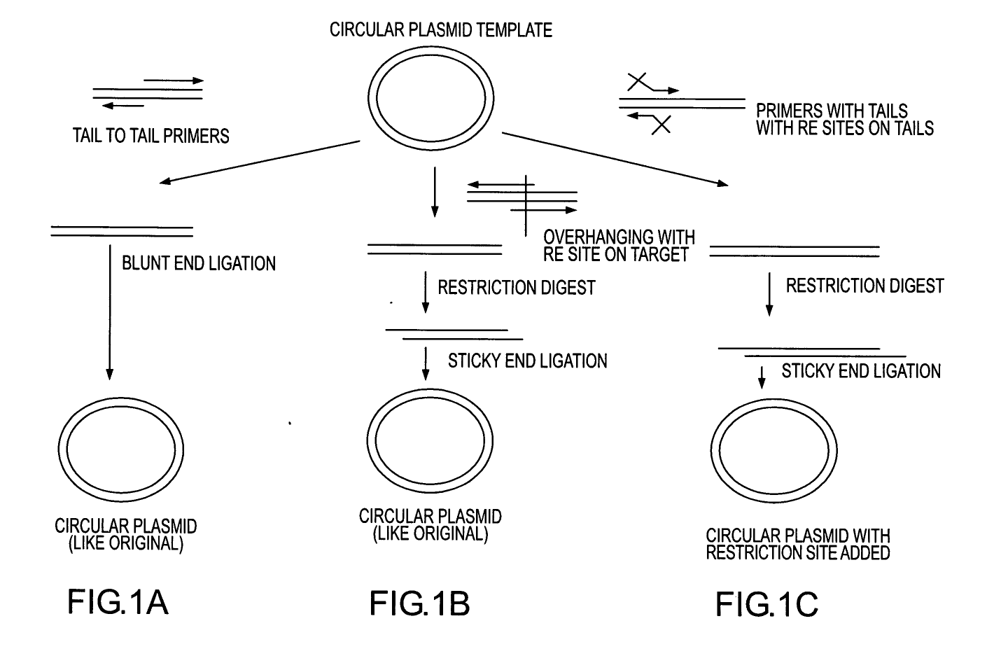

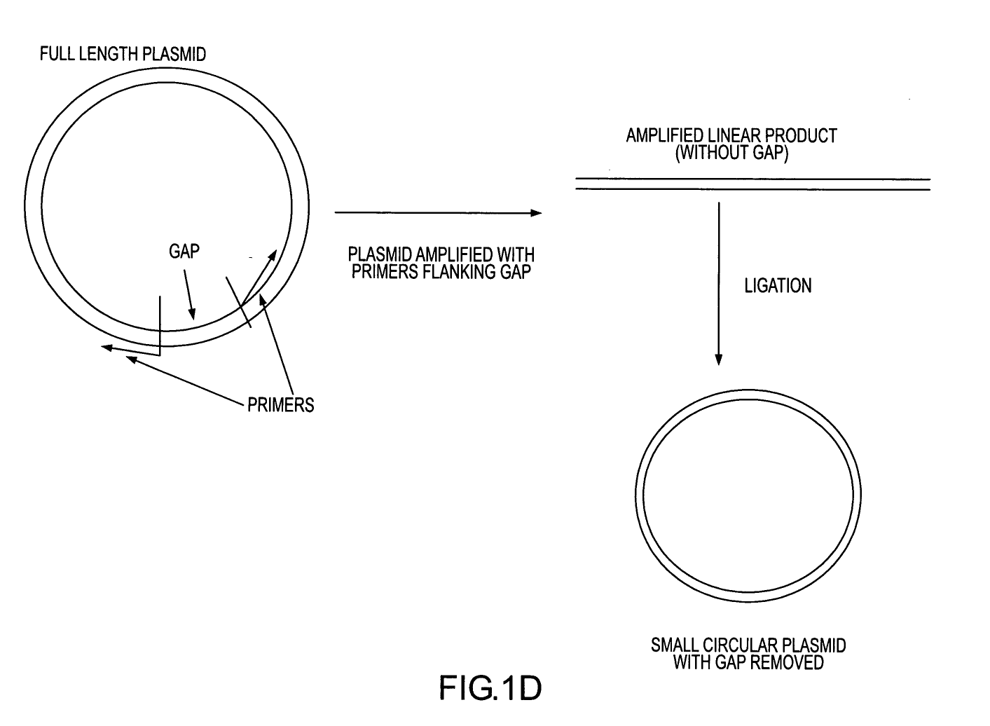

In one embodiment, the 5′ end of each primer hybridizes to adjacent base pairs on opposite strands of the template leaving a zero-gap or no-gap configuration, as illustrated in FIG. 1A. This particular embodiment is useful for the amplification of entire episomal genetic elements that are circular in nature, i.e., plasmids, cosmids, BACs, and the like. In another embodiment, the 5′ ends of each primer can overlap on adjacent strands of the extrachromosomal DNA elements so that additional sequence of each strand of the template is amplified, as illustrated in FIG. 1B. In an alternative embodiment, shown in FIG. 1C, one or both primers may include a non-complementary extension of the 5′end for introducing additional sequence into the amplicons. In a further embodiment, a gap can be introduced between the 5′ends of each primer on adjacent strands of the episomal genetic element, thereby eliminating the sequence between the 5′ end of each primer from the amplification product. See FIG. 1D. Thus, using the compositions and methods provided herein, amplification reactions can be performed to maintain the original extrachromosomal nucleic acid sequence or can be add or remove target sequences from the resulting amplicons. Preferred embodiments envision that the entire extrachromosomal nucleic acid be amplified.

Primer design having a zero-gap configuration (FIG. 1A) is generally preferable where an exact sequence of an amplification product of the template is desired, however, a gap configuration can be used where a target sequence is not required in the amplification product (FIG. 1D), i.e., where a selection marker or other target sequence is no longer required. The overlapping design is generally used where the additional sequence that is amplified provides a restriction endonuclease site for introducing the site at the end or ends of the target template episomal genetic element (FIG. 1B).

In a further and preferred aspect, the invention provides universal primers. As used herein, universal primers refers to standardized primers complementary to a highly conserved region within a target DNA, e.g., closed, circularized nucleic acids having a highly conserved region in the circular template molecule, for example the origin of replication sequence for plasmids. Note that universal primers of the invention are useful in the amplification of plasmids, cosmids and other higher molecular weight extrachromosomal nucleic acid molecules as long as the episomal genetic element has a common target sequence, for example a common origin of replication sequence. Illustrative examples of universal primer targets for amplification of nucleic acid molecules include: promoter sequences found in a majority of vectors, drug resistance genes found in a majority of vectors, e.g., the ampicilin gene (Amp), the cos site found in cosmids and lambda phage, the origin of replication in bacteria, and highly conserved regions of any of the mitochondrial genes found in mitochondrial DNA. Table 1 provides illustrative sites for universal primer design in accordance with the present invention.

TABLE 1
Target Hybridization Sites For Universal Primer Design

| Universal Primer Target Hybridization Site | Organism |
|---|---|
| Ampicillin Resistance Gene | Bacteria |
| 2 Micron Origin | Yeast |
| Kanamycin Resistance Gene | Bacteria |
| Zeocin Resistance Gene | Bacteria |
| Lambda Cos Site | Cosmids and Phage |
| Origin of Replication | Bacteria |

The following description is focused on an illustrative universal primer designed for a target template within a bacterial origin of replication. Universal primers of this embodiment include any primer having from about 10 to about 75 contiguous base pairs, and preferably from about 15 to about 40 contiguous base pairs, and most preferably from about 20 to about 30 contiguous base pairs complementary to a portion of the origin of replication sequence for the plasmid of interest.

Note that these same primer lengths are generally useful in the design of other universal primers, i.e., non-origin of replication. Surprisingly, the origin of replication of plasmids is an excellent target for universal primer design. For example, the approximate 668 base-pair Co1E1 origin of replication is present in over 90% of all commonly used plasmids. Universal primers can be designed to hybridize anywhere within that 668 bp region (Table 2, SEQ ID NO: 1). In addition to plasmids having the Co1E1 origin of replication, other important plasmid families or groups include the “phage type” plasmids, e.g., lambda, p13, p10, also having a conserved origin of replication.

TABLE 2
ColE1 Origin Of Replication (Sense Strand) (SEQ ID NO:1)

5′-TGAGCAAAAG GCCAGCAAAA GGCCAGGAAC CGTAAAAAGG CCGCGTTGCT
GGCGTTTTTC CATAGGCTCC GCCCCCCTGA CGAGCATCAC AAAAATCGAC
GCTCAAGTCA GAGGTGGCGA AACCCGACAG GACTATAAAG ATACCAGGCG
TTTCCCCCTG GAAGCTCCCT CTGGCGCTCT CCTGTTCCGA CCCTGCCGCT
TACCGGATAC CTGTCCGCCT TTCTCCCTTC GGGAAGCGTG GCGCTTTCTC
ATAGCTCACG CTGTAGGTAT CTCAGTTCGG TGTAGGTCGT TCGCTCCAAG
CTGGGCTGTG TGCACGAACC CCCCGTTCAG CCCGACCGCT GCGCCTTATC
CGGTAACTAT CGTCTTGAGT CCAACCCGGT AAGACACGAC TTATCGCCAC
TGGCAGCAGC CACTGGTAAC AGGATTAGCA GAGCGAGGTA TGTAGGCGGT
GCTACAGAGT TCTTGAAGTG GTGGCCTAAC TACGGCTACA CTAGAAGGAC
AGTATTTGGT ATCTGCGCTC TGCTGAAGCC AGTTACCTTC GGAAAAAGAG
TTGGTAGCTC TTGATCCGGC AAACAAACCA CCGCTGGTAG CGGTGGTTTT
TTTGTTTGCA AGCAGCAGAT TACCGCGCAG AAAAAAGGAT CTCAAGAAGA
TCCTTTGATC TTTTCTAC-3′

Preferably, in one embodiment, the forward and reverse primer are designed to hybridize in tail-to-tail fashion (i.e., 5′-end to 5′-end) on opposite strands within the origin of replication (See Table 1 above for sequence of origin of replication). In preferred embodiments, the primer pairs are configured to hybridize to adjacent base pairs, i.e., have zero base pairs intervening between the 5′ end of the forward primer when hybridized to the sense strand and the 5′ end of the reverse primer when hybridized to the antisense strand of the template plasmid DNA. In alternative embodiments, the two primer tails can overlap any number of primer-derived base pairs across the dsDNA. Overlap configurations, as discussed in more detail below, present excellent restriction endonuclease targets within the amplified double-stranded amplicons of the invention or alternatively primer extensions can be used to introduce restriction sites or other useful sequences into the resulting amplicons. For example, an extended primer tail can include the restriction site for a rare restriction enzyme, thereby incorporating the site into the amplicon.

In more detail, the design of optimal universal primer pairs is based on selecting primer sequences within a highly conserved region, for example the origin of replication of a target plasmid, for example, within the 668 base pair origin of replication for Co1E1 based plasmids. Typical primer designs for plasmids have tail-to-tail (i.e., adjacent 5′ ends) designs within a double stranded template region of from about 20 bp to about 150 bp. Each primer of the primer pair anneals to an opposite strand of the double-stranded plasmid molecule, and generally is from 15 to 40 bp in length. Note that longer primer molecules can be used in embodiments of the present invention, but these molecules are of limited value beyond the 40 base pair primer, due to minimal or detrimental effects on stability and specificity.

Initial parameters for designing universal primers within the origin of replication, for example, generally include finding a unique conserved region within the origin of replication. Proposed primer pairs are analyzed using a primer design program, for example Vector NTI, (InforMax, Golden, Colo.), for appropriate Tm, palindromic sequences, −ΔG, and the like. Secondary structure is co-analyzed using a secondary program, for example, Visual OMP, (DNA Software, Ann Arbor, Mich.). Primer designs having appropriate Tm, lower secondary structure, lower likelihood of multimer formation and the like are then selected for further testing on target plasmids.

Primer pairs having appropriate designs are empirically utilized for capacity to prime and elongate complementary plasmid strands. Plasmid primer pairs are designed to anneal within the highly conserved region, e.g., origin of replication, under the buffering conditions of the present invention (i.e., lower salt concentrations, lower overall ionic strength, and inclusion of agents to stabilize double-stranded regions of nucleic acid). Further, primer pairs can be designed to stably anneal on the template sequence at the same temperature that the polymerase amplifies off of the annealed primer. This allows for a two-step amplification cycle, as opposed to a three-step amplification cycle, which no longer requires a different primer annealing temperature. This is particularly important for enhanced specificity, where primers of the present invention anneal at the higher temperatures and thereby avoid lower temperature non-specific binding. Further, a two step amplification cycle is considerably less time consuming and results in less non-specific product formation.

In an alternative embodiment, forward and reverse primers are additionally designed to overlap at their 5′ends on opposite strands within the common region, and in particular to overlap at their 5′-end sufficiently to create a restriction digest site on the amplicons to produce, under appropriate enzyme activity, a sticky end. For example, an amplicon amplified from four base-pair overlapping primers would have four base-pair sticky ends for enhanced ligation. As an illustrative example, restriction sites for several restriction enzymes are located throughout the 668 bp region of the origin of replication. One example is the Drd I ten-mer site. The plasmid pair would be designed to include an overlap of the 10 bps that represent the Drd I restriction site. Amplified product from this overlap primer configuration would be treated with Drd I to prepare consistent overhangs at the 3′ and 5′ ends of the double-stranded amplicon (assuming the particular enzyme can cut at the end of the amplicon). In some embodiments, the primer pairs are designed to overlap so that there are two or three non-specific extra bases 5′ to the recognition sequence.

In general, the primers of the present invention are designed to be complementary to adjacent target sequences on the circular nucleic acid template, such that hybridization may occur during the annealing steps of the cycling reactions. As such, complementarity need not be perfect; there may be any number of base-pair mismatches that will interfere with hybridization between a primer sequence and its corresponding target sequence. However, if the degree of non-complementarity is so great that hybridization between a primer and its target cannot occur under even the least stringent of conditions, the primer sequence is considered to be not complementary to the target sequence. Thus, “substantially complementary” herein is meant to describe a situation where a primer sequence is sufficiently complementary to the corresponding region of its target sequence to specifically hybridize under the selected reaction conditions.

The following description illustrates several of the parameters necessary for proper design of origin of replication primer pairs. Hybridization generally depends on the ability of primers to anneal to single-stranded nucleic acid strands bearing substantially complementary sequences in an environment near the melting temperature of the hybridized adduct. Note that in general, the higher the degree of complementarity between the pair of DNA sequences (primer and template), the higher the relative temperature that can be used while maintaining the potential for hybridization, assuming other factors are held constant.

Generally, the length of a primer and its GC content will determine the thermal melting point (Tm) of a hybridization complex, and thus contribute to defining the hybridization conditions necessary for obtaining specific hybridization of a primer and a template. These factors are well known to a person of skill in the art, and can be tested experimentally. The Tm is the temperature (under defined ionic strength and pH) at which 50% of the hybridization complex exists between primer and template. Hybridization of nucleic acids is well within the purview of the skilled artisan and an extensive guide is found in Sambrook et al., supra. Stringent conditions are generally defined to be at or above the Tm for a specific sequence at a defined ionic strength and pH. Typically, G−C base pairs should be maintained at a reasonable content. For example, a 20 base pair primer with approximately 50% G+C content has Tm values between about 56° C. to 62° C. More sophisticated models for Tm are available, and their application may be more appropriate if G−C stacking interactions, neighboring nucleotides, solvent effects, and the like need to be considered.

In general, specificity of the universal primers with target template molecules is controlled by primer length and annealing temperature of the amplification reaction. The primers that bind to the circular nucleic acid will generally be composed of naturally occurring nucleotides, but in some cases may contain one or more nucleotides bearing unnatural features such as a modified sugar unit or an absence of a sugar unit; a modified sugar-phosphate chain where one or more oxygen atoms of the phosphate are substituted by sulfur, carbon, and/or nitrogen atoms, or the like; a modified or an absence of a base unit; or other modification that can provide synthetic advantages, stability under the conditions of the assay, resistance to enzymatic degradation, etc. In one embodiment, modified nucleotides that do not significantly impact the Tm values are incorporated into the primers. In other embodiments, modified nucleotides do impact the Tm, allowing for the design of shorter primer length with maintenance of corresponding specificity.

Universal primer polynucleotide molecules of the invention can also include chemically modified bases that resist enzymatic degradation. For example, universal primers of the present invention can be designed to incorporate modifications for resisting degradation via endonuclease activity. In one embodiment, universal primers can be modified to resist degradation by the proofreading activity of a polymerase thereby allowing the universal primers to be stored in the same solution as the polymerase enzyme, i.e., the universal primers can be modified to resist degradation by a 3′-5′ exonuclease proofreading activity associated with a number of proofreading DNA polymerases (Vent, Pfu, Klenow fragment and T7 DNA polymerase).

Illustrative examples of primer modifications include: incorporating one or more chemically modified bases, for example a primer designed to have a LNA at the n-1 position (See Examples and Di Giusto et al., (2004) Nucleic Acid Research, 32(3):e32). Additional illustrative modifications are shown in Table 3. In several of the embodiments shown in Table 3, the primer is reversibly modified to block proofreading nuclease activity, rendering the primer non-extendable. The modification is removed when the universal primer of the invention is ready for use in an in vitro amplification reaction of the present invention via an appropriate reversal technique (See Table 3).

TABLE 3
Primer Modifications For Increased Stability In Presence of
Proofreading Activity

| | | Unblocking Mechanism For |
|---|---|---|
| | | Non-Extendable |
| Modification | Extendable/Non-Extendable | Modifications |
| LNA at n-1 position | Extendable | |
| 3′-thiophates at n-1, n-2 or n-3 | Extendable | |
| 3′-terminal phosphorylation | Non-Extendable Under | Cleave phosphate group with |
| With Hypothermophilic | Storage Conditions | Endo-3 or Endo-4 derived |
| Phosphorylase | | from a thermophilic organism, |
| | | e.g. Thermus thermophilus, |
| | | Thermotoga maritima, and the |
| | | like. |
| A-Basic Phophoamidite | Non-Extendable Under | |
| | Storage Conditions | |
| Biotinylated Nucleotide at or | Non-Extendable Under | Heat release of streptavidin |
| near 3′-end | Storage Conditions In | from biotinylated nucleotide |
| | Presence of Streptavidin | |

Solutions used in conjunction with the primers will be discussed in more detail, however solutions are provided in the invention for annealing of the primer to the extrachromosomal DNA, for example the primer to the origin of replication of plasmid DNA.

Primer design within an origin of replication of a target plasmid family, using the parameters discussed above, is translated into appropriate length oligonucleotides. Composition of these oligonucleotides can be through the use of chemically modified nucleic acid or simply through the use of non-modified nucleic acid molecules. In either case, the prepared oligonucleotides of the invention can be stored for use with plasmids having the target origin of replication. In this manner, the primer pairs are universal for a target class of plasmids that share the same origin of replication. This same inventive rationale can be used to design alternative universal primer pairs for numerous template targets.

As discussed above, although not universal in nature, it is also envisioned that primers can be designed for use with target template molecules that do not possess an origin of replication. These primers could be designed to prime target regions of the target template as is known in the art. The same general parameters of design would apply as discussed above.

Polymerase Reaction Buffer

Embodiments of the present invention include compositions that support in vitro amplification of nucleic acid molecules, for example episomal genetic elements, including amplification of closed, circular nucleic acid molecules in cultured cells, i.e., plasmid DNA, cosmid DNA and the like. In preferred embodiments, the nucleic acid molecule is typically about 1 kb to 40 kb, and more typically about 2 kb to about 25 kb and can be from about 2 kb to about 20 kb. Amplification reaction buffers for use in the present invention generally have low ionic strength, as compared to conventional long range PCR reaction buffers, typically having a pH range of from 8.0 to 9.0, and preferably from 8.8 to 8.9. In addition, compositions of the present invention typically only require small concentrations of co-solvent, i.e., much lower concentrations of co-solvent than conventionally used in long range PCR, for example lower concentrations of DMSO, due to the low ionic strength of the reaction buffer. The lower co-solvent requirement allows for compatible enzyme stability (due to the low ionic strength of the buffer) but avoids the negative co-solvent issues, such as lower enzyme fidelity in the presence of the solvent. Further, compositions of the present invention generally provide Mg²⁺ buffering characteristics, allowing for optimal polymerase activity in a wide range of Mg²⁺ ion concentrations.

Embodiments of the present invention also provide solutions that include 10 to 150 μM weak organic base, 10 to 50 μM weak organic acid, 1 to 4 mM magnesium acetate, 2 to 20 mM (NH)₂SO₄, 50 to 200 μM dNTPs, and 0.1 to 5 units or more thermophilic polymerase. Preferred embodiments include one or more polymerase enhancers, for example, trehalose, sorbital, DMSO, or a mixture of sorbital, trehalose and/or DMSO, especially if the target amplification target is “GC” rich. Note that DMSO, and other like solvents, when included at all, are kept to a minimum concentration, typically less than 1% (conventional buffers include about 5% co-solvent). Also note, as briefly discussed above, the use of weak organic acids and weak organic bases to buffer the pH to between 8.9 and 9.0 eliminates the propensity of increasing the solutions ionic strength, and eliminates the need for higher concentrations of co-solvents, thereby maximizing the fidelity of the polymerase. In addition, utilization of particular weak organic acids can prove to be extremely effective at buffering Mg⁺⁺ concentrations within the preferred Mg⁺⁺ concentration of 1.5 to 8 mM range for most amplification reactions. In particular, use of bicine within the concentration ranges discussed herein proves to be excellent at buffering Mg⁺⁺ concentrations (bicine is a Mg⁺⁺ chelator).

Preferred buffer embodiments of the present invention typically include a combination of a weak organic base and a weak organic acid, combined in amounts to provide a pH of about 8 to 9, preferably about 8.80 to 8.90, and most preferably about 8.85.

In another preferred embodiment of the present invention, the buffer comprises a combination of 30 mM bicine, 200 mM tris, 4-10 mM (optimal is 6 mM) ammonium sulfate, 2.5 mM magnesium ions, pH 8.85 to 8.90.

In one embodiment the thermal cycling condition in the present invention includes a three-step cycle process—a denaturation phase, an annealing phase and an extension phase. The thermal cycling conditions can include an initial denaturation step or enzyme activation step, for example, hot start PCR, at the start of each amplification reaction (U.S. Pat. No. 6,667,165 incorporated herein by reference in its entirety). Temperature ranges for cycling conditions are dependent on length of template nucleic acid, GC content of template, length and sequence of primers and co-solvents. In one embodiment the initial denaturation condition is for about 5 minutes, followed by a denaturation step for 10 seconds to one minute at about 90-95° C., followed by an annealing step for about twenty seconds between 45 and 70° C., followed by an extension step that lasts for one to ten minutes at about 65 to 75° C. The cycle can be repeated twenty to forty times.

Embodiments of the present invention include a two-step thermal cycling process: The process includes a denaturation step that lasts ten seconds to one minute at about 90-95° C. followed by an annealing and extension step that lasts about one to ten minutes at 60 to 72° C. An initial denaturation step can be included as above (approximately five minutes). Primers for use with this embodiment are designed to anneal and extend at the same temperature. The steps can be repeated twenty to forty times.

In addition, thermal cycling is extremely effective/efficient for direct in vitro amplification of plasmid DNA (as well as for other episomal genetic elements) from a host cell. Table 4 illustrates a typical combination of ingredients for an embodiment of the in vitro amplification buffer for a target extrachromosomal DNA for example an episomal genetic element, i.e., a pUC19 plasmid.

TABLE 4
Amplification Reaction Buffer

| | | Concentration | Preferred |
|---|---|---|---|
| Generic Ingredient | Illustrative Example | Ranges | Concentration |
| Thermophilic | Taq; Tth; Pfu; Tma; | 0.25 to 5 or more | 0.5 units in a 10 μl |
| Polymerase | and the like and | units | reaction mix |
| | combinations thereof | | |
| DNTPs | dATP, dCTP, dTTP, | 50-200 μM | 100 μM |
| | dGTP | | |
| weak organic acid | Bicine, Tricine, | 10 to 50 mM final | 20 to 40 mM final |
| | Tapso, CHES, and | reaction | reaction |
| | combinations thereof | concentration | concentration |
| weak organic base | tris, bis-tris-propan, | 10 to 150 mM final | 50 to 70 mM final |
| | and combinations | reaction | reaction concentration |
| | thereof | concentration | |
| reaction enhancer | sorbitol, trehalose, | 0.5 to 3% DMSO, | 200 mM sorbitol |
| | DMSO, glycerol, | | 200 mM trehalose |
| | mannitol, and | | 0.6% DMSO |
| | combinations of the | | |
| | above | | |
| Salt | ammonium sulfate | 4 to 20 mM final | 4 to 12 mM final |
| | | reaction concentration | reaction concentration |
| Mg²⁺ | magnesium acetate | 2 mM to 5 mM | 2 mM |

The embodiments of the present invention support the amplification of extrachromosomal DNA, i.e., plasmids, directly from purified DNA, from glycerol stocks, and from colony picked cells. In some embodiments the colony picked cells are re-suspended in a small volume of water (5 to 15 μl) or added directly to the PCR buffer mixes of the present invention.

As indicated within Table 4, a large number of thermophilic polymerase enzymes can be used to amplify the target nucleic acid. The low ionic strength of the amplification buffer, and minimal level of co-solvent(s), allows for the use of traditionally ineffective long range PCR thermophilic polymerase enzymes. As such, most thermophilic polymerase I enzymes can be used in the context of the present invention, including Taq, Tth, Pfu, and Tma. Thermophilic polymerase III enzyme systems can also be used to amplify template DNA, for example using the enzyme subunits described in U.S. Pat. No. 6,238,905, or U.S. Pat. No. 6,555,349, which are both incorporated herein in their entirety. One thermophilic polymerase for use in the present invention is sold under the brand name Eppendorf Triple Master Enzyme.

Embodiments of the present invention provide methods for manipulating amplified nucleic acid molecules into closed, circular, nucleic acid molecule products. After the amplification reaction is complete, and the appropriate amounts of product have been produced, a ligation reaction can be performed to circularize a percentage of the product into closed circular DNA.

In one embodiment, molecules amplified using the methods of the present invention can be engineered to exhibit complementary “sticky” ends incorporated at each end of the product. The addition of sticky ends to an amplified nucleic acid molecule can be accomplished via any one of several known techniques, i.e., digestion with restriction enzyme or ligation of matching corresponding oligonucleotide at each end. The amplified products having the sticky ends are then circularized via a standard ligation reaction using standard ligase reaction conditions. The ligase can be non-thermophilic or thermophilic. Note that a number of bases are added to the plasmid in this embodiment to form the “sticky end.” Typically, high molecular weight templates include a portion or all of the sequence for a plasmid, cosmid, BAC or YAC, preferably of a plasmid, so that the amplification/ligation reactions replace standard, in vivo derived, preparation and purification procedures.

In an alternative embodiment, a unique restriction enzyme site is located within the target plasmid. The restriction site is preferably present only once within the sequence of the target plasmid, and primers are designed to overlap the restriction enzyme recognition site. The amplicon produced is then treated with the appropriate restriction enzyme to product sticky ends within the designed restriction site. The amplicon sticky ends allow subsequent ligation reactions to produce circular product.

The reactions outlined herein may be accomplished in a variety of ways, as will be appreciated by those skilled in the art. Components of the reaction may be added simultaneously, or sequentially, in any order. In addition, the reaction may include a variety of other reagents, including additional buffers, innocuous proteins (e.g., albumin), detergents, etc., that may be used to facilitate optimal primer annealing and amplification, and/or reduce non-specific interactions. Also, reagents that otherwise improve the specificity or efficacy of the assay, such as protease inhibitors, nuclease inhibitors, anti-microbial agents, etc., may be used, depending on the sample preparation methods and purity of the target.

Polymerization and Ligation Enzymes in one Reaction Mix:

In an alternative embodiment, the polymerase and ligase enzymes are included in the same reaction buffer, and nucleic acid is amplified and ligated within the same reaction buffer. Typical reaction pH for optimal one-step polymerization/ligation is about 7 to 9, and preferably from about 8 to 9, and most preferably from about 8.3 to about 8.6. Table 5 illustrates a typical combination of ingredients for an embodiment where the amplification and ligation reactions are performed substantially simultaneously:

TABLE 5
Combined Amplification and Ligation Reaction Buffer

| | | Final | Preferred Final | |
|---|---|---|---|---|
| | | Concentration | Concen. (50 μl | |
| Ingredient | Example | Range | reaction volume) | |
| dNTPs | Mixture of dCTP, | 200 to 700 μM | 500 | μM |
| | dTTP, dGTP and | | | |
| | dATP | | | |
| Thermophilic | Tth, Taq, | 0.5 to 5 | 2.5 | units |
| Polymerase | | units/50 μl | | |
| | | reaction | | |
| | | volume | | |
| Ligase | Tsc, Pfu, | 1.5 to 20 ngl | 5 | ng of ligase |
| | | ligase/50 μl | | |
| | | reaction | | |
| | | volume | | |
| Organic Base | Bicine, Tricine, | 10 mM to 150 | 30 | mM |
| | CHES, TAPSO | mM | | |
| | and combinations | | | |
| | thereof | | | |
| Organic Acid | Tris, Bis-tris- | 10 to 50 mM | 20 | mM |
| | propane, and | | | |
| | combinations | | | |
| | thereof | | | |
| Salt | (NH)₂SO₄, | 1 mM to 10 | 6 | mM |
| | NH₄Cl | mM | | |
| Mg⁺⁺ | Mg(Oac)₂ | 2 to 8 mM | 5 | mM |
| Energy Source | β-NAD | 0.1 to 5 mM | 1 | mM |

Polymerase for use in the amplification and ligation buffer are as described above and include most polymerase combinations useful in conventional long range PCR reactions, for example, polymerase I and a proofreading enzyme. Note also that polymerase enzymes from pol III, preferably pol III from a thermophilic species, may also be used within the methods of the present invention. Specific polymerase enzymes for use in the invention include polymerase I from Thermus thermophilus, Thermus acquaticus, and Thermotoga maritima (particular products for use include Eppendorf Triple Master Enzyme, ABI RT-PCR kit enzyme, and Roche Expand enzyme kit). Ligase enzymes useful in the present invention include thermophilic ligase enzymes, for example, Tsc ligase (Prokaria, Product No. Dllg 119). Finally, as noted above, about 0.4 μM of each universal primer is included in the reaction with the template of choice where the nucleic acid is a plasmid or other like compound. Optionally, about 1 mM DTT or other like agent can be added as a reducing agent. As above, polymerization enhancers may also be added to the reaction in similar concentrations as described in the previous embodiment.

Thermal cycling parameters are those typically as described above. In some embodiments a ligation step, incubation time, and temperature can be included. In one preferred thermal cycling embodiment, the cycling parameters include an initial denaturation step (93° C. for two minutes), followed by heating to 94° C. for twenty cycles of 20 second 94° C. heating steps, followed by a final cycle of 68° C. for three and one-half minutes. The plurality of cycles above is typically from 20 to 30 in number, preferably from 25 to 30 in number.

Reaction efficiency varied for polymerase/ligase reactions, but at least about 5% or more of the amplicon can be converted into ligated, covalently-closed, circular DNA product during the reaction.

Assays Incorporating the Methods and Compositions of the Present Invention

Embodiments of the invention provide assays that incorporate the primers, amplification reaction buffers, and direct in vitro amplification and ligation methods. Target template DNA for methods of the present invention include extracted extrachromosomal DNA, bacterial colonies, picked DNA and bacterial colonies stored in glycerol stocks.

In one embodiment of the invention, in vitro amplification of plasmids directly from target host cells is provided. This process can replace the need for replicating plasmid DNA within a host cell, i.e., miniprep, maxiprep, etc. The method includes picking the target colony cells that harbor the plasmid DNA of interest. The cells are either re-suspended in water (or some equivalent aqueous medium) or directly provided to the reaction mix. The amplification reactions of the present invention are as described above. Amplicons can be circularized either in a subsequent reaction, as described using the polymerase reaction buffer, or within the same reaction using the combined amplification/ligation reaction buffer (both as described above). Circularized product as formed using the methods and compositions of the present invention can be isolated using any one of a number of well known procedures (for example, see Maniatis). In this manner, high fidelity amplification of plasmid DNA can be accomplished, thereby alleviating the need for the in vivo preparation and purification of plasmid DNA.

In an alternative assay, the methods and compositions of the present invention can be used to identify specific extrachromosomal DNA molecules. Importantly, compositions and methods of the invention can be used to amplify and thereby test for unique plasmid sequences found in target organisms, for example, use of the compositions and methods of the invention to identify sequences found only in specific cell types. In particular, target cells are collected and cracked with heat treatment (where required) and any present extrachromosomal DNA amplified using the primers and compositions of the invention. Amplified extrachromosomal DNA can be accurately sized, sequenced or tested for the tell-tale genetic fingerprint associated with a specific marker. The method of identification can streamline screening processes and will remove the need for growing the “unknown” organism, which takes time and specialized equipment.

In another embodiment of the invention, the same methods and compositions can be used to identify large target sequences, i.e., 10 to 20 kb, within the cells removed during a biopsy of tumor cells, for example, primers used within the 3′ and 5′ ends of the p53 gene, amplify the entire sequence of the p53 gene instead of specific “hot spots” and sequence the gene to provide a map of the patient's entire gene. Target oncogenes and tumor suppressor genes would be associated with diagnosed tumors. These embodiments do not rely on the use of universal primers, but rather, on the high fidelity of the inventive buffers of the present invention. Data obtained from the analysis would then be used as a prognosticator of the patient's survivability as well as to provide additional information as to why certain cells lost regulation over their growth. Alternatively, a series of long range, high fidelity PCR can be performed on “healthy cells” to determine whether a subject who has not been diagnosed with cancer has indicative mutations within certain known oncogenes and tumor suppressor genes. Again, this information would be useful in the context of a clinical setting. This type of long range high fidelity PCR can be used within the context of numerous clinical settings for the identification of viral agents, bacterial agents, mycoplasma, etc.

In another embodiment, long range PCR methods and compositions of the present invention can be used to determine an individual's haplotype as specific series of polymorphic sites within a parentally derived chromosomal region by redesigning the PCR primers of the invention. (Note that like the previous embodiment, the inventive features do not require universal primer pairs or extrachromosomal DNA elements.) Using the enhanced processivity and fidelity of PCR embodiments of the present invention, yields of quality, large molecular weight, polymorphic site amplicons are obtained and can be reliably sequenced, providing valuable information for correct haplotyping. Application of haplotype will be useful in medical diagnosis and prognosis as genomic therapeutics are discovered and tested during clinical trials.

In another embodiment, the methods and compositions of the invention are used to prepare adequate amounts of target extrachromosomal DNA for sequencing. For example, amplicons prepared using the methods and compositions of the present invention can be directly sequenced (See Example 4) using standard sequencing protocols on large, insert containing, plasmid vectors. Further, intermediary gel analysis can be performed on amplicons to determine the presence of a plasmid prior to sequencing. These embodiments streamline and provide higher quality to the process.

In yet another embodiment, the methods and compositions of the invention are used to facilitate DNA cloning applications. For example, in vitro amplified plasmids of the invention can be utilized to perform recombinant DNA procedures without having to culture, select and/or purify extrachromosomal DNA from bacterial cells.

Amplification and Amplification and Ligation Kits

Embodiments of the present invention provide kits for the performance of the above-described nucleic acid amplification methods. Kits can be directed toward generation of linearized, extrachromosomal DNA, or directed toward simultaneous amplification and ligation yielding circularized extrachromosomal DNA products.

In one embodiment of the present invention, the kit(s) include, in one or more tubes, the buffering compositions, amplification reactants and, optionally, ligase reaction mixes necessary to practice the present invention. A preferred embodiment of the kit may further include universal primer pairs of the present invention, either contained in a separate tube or integrated into the appropriate reaction mix above. The primer pairs can be sold in kits that are specified for a particular type of extrachromosomal DNA target of interest, e.g., ColE1 origin of replication-based plasmids, two micron-based origin of replications, and p10 like origins of replication. Similar arrangements can be applied to yeast-based, BAC-based or other like system. The kits of the present invention can optionally include the oligonucleotides required for incorporation of a sticky end(s) to the amplified product, as well as molecular grade water, collection tubes, agarose or other detection like material, 96 and other multi-well plates, pipette tips, PCR cyclers, microcentrifuge, protective gloves, safety glasses, etc.

Alternatively, kits can include a combined amplification and ligation solution of the invention i.e., within the same tube. For example, the kits could include all of the ingredients as described above except that the reaction buffer would be a reaction buffer consistent with the amplification and ligation reaction buffer of the invention.

In some embodiments, the kit could also include restriction endonuclease enzyme(s), for use in testing a sample of the reaction product to determine if the amplicon is a closed-circle nucleic acid or linear nucleic acid. The kit could also include restriction endonuclease enzymes for embodiments that include universal primers having incorporated endonuclease restriction sites wherein the enzyme and site would correspond within each kit.

For maximum stability, the kits can contain lyophilized ingredients where appropriate.

Having generally described the invention, the same will be more readily understood by reference to the following examples, which are provided by way of illustration and are not intended as limiting.

### EXAMPLES

### Example 1

### Identification of Universal Primers For Amplification of Plasmid DNA

Sequence alignment and comparisons for a number of commonly used plasmids, e.g., pGEM, pUC18, pUC19, pBR322, etc, were performed using the BLAST alignment program within the NCBI database. An examination of the sequence alignment data revealed a region of identity located within a majority of the examined plasmid sequences, the region being approximately 668 base-pairs in length. A determination of sequence identity compared to known sequences was performed, showing that the sequence corresponds entirely to the sequence of the ColE1 origin of replication (See Table 2). Upon further investigation, it was determined that the plasmids that contained this region of identity were in fact plasmids derived from the E. Coli natural plasmid, ColE1. Table 6 provides a partial list of plasmids that include the ColE1 origin of replication:

TABLE 6
ColE1 Origin of Replication Containing Plasmids

| | | | |
|---|---|---|---|
| pUC18 | pUC19 | pEGFP | pET |
| PGEM | pBS | pCR-TOP0 | pGEX |
| PQE | pPICZ | | |

Universal primers, in accordance with the present invention, were designed to hybridize to a site within this region of high identity. Initially, primers were designed to hybridize to a sequence of bases within the origin of replication (See Table 2). Primer design took into account the temperature and ionic strength parameters of the present invention shown to be useful in long range colony PCR. Forward and reverse primers were designed to hybridize tail-to-tail (5′ ends of each primer located on adjacent, opposite, strand bases and directed in opposite orientation—forward and reverse) to ensure that the entire plasmid sequence was amplified during the amplification reaction.

Because the design of universal primers for in vitro amplification reactions reduces the time and cost of having to engineer individual primer pairs for each target plasmid, and ensures that the entire sequence of the plasmid is amplified, further investigation was performed to optimize primer pairs. Universal primers were modified in their length and hybridization position within the origin of replication. Table 7 illustrates several primer pair designs for use in the present invention (note that the same rationale applies to other plasmid families, for example, using the origin of replication sequence for plasmids that were derived from phages, for example, lambda, p13, p10, etc. (See Table 1)).

TABLE 7
Illustrative Primer Pairs For Use In Present Invention

| | | |
|---|---|---|
| Primer Pair ID | Sense Primer | Antisense Primer |

| | | |
|---|---|---|
| AR Orig ORI | cactgattaagcattggtaactgtc | aggcacctatctcagcgatctgtc |
| DNA long | cactgattaagcattggtaactgtctatttc | aggcacctatctcagcgatctgtcagacc |
| LNA-9version | cactgattaagcattggtaactgtc | aggcacctatctcagcgatctgtc |
| UniOri 20 | ctgaagccagttaccttcgg | cagagcgcagataccaaat |
| UniOri 22 | ctgaagccagttaccttcggaa | cagagcgcagataccaaatact |
| UniOri 25 | ctgaagccagttaccttcggaaaaa | cagagcgcagataccaaatactgtc |
| UniOri 30 | ctgaagccagttaccttcggaaaaagagtt | cagagcgcagataccaaatactgtccttct |

Experiments were designed to identify and test potential universal primer pairs for capacity of amplifying a target plasmid. Using the identified primer region above, potential primer pairs were designed having 20, 22, 25, or 30 bps in length and submitted for analysis through a combination of Vector NTI and Visual OMP programs. Primer pairs were designed to sit 5′ end to 5′ end (in opposite orientation) on adjacent strands, having no bases between or overlapping each primer sequence. Amplification reactions were performed using 0.3 μM of each pair of primers in a two-step cycling reaction (95° C. and 68° C.), utilizing the inventive long range PCR buffer conditions described herein, in particular, 30 mM bicine tris, 6 mM (NH₄)SO₄, 200 μM dNTPs, 2.5 mM Mg²⁺, enhancers (combination of DMSO, sorbitol, and trehalose), and 2.5 units Triplemaster Enzyme, at a pH of 8.9.

| | | |
|---|---|---|
| 20mer (s) | (SEQ ID NO: 2): | 5′-ctgaagccagttaccttcg |
| | | g-3′ |
| 20mer (as) | (SEQ ID NO: 3) | 5′-cagagcgcagataccaaa |
| | | t-3′ |
| 22mers (s) | (SEQ ID NO: 4): | 5′-ctgaagccagttaccttcg |
| | | gaa-3′ |
| 22mer (as) | (SEQ ID NO: 5) | 5′-cagagcgcagataccaaat |
| | | act-3′ |
| 25mers (s) | (SEQ ID NOs: 6): | 5′-ctgaagccagttaccttcg |
| | | gaaaaa-3′ |
| 25mers (as) | (SEQ ID NOs: 7): | 5′-cagagcgcagataccaaat |
| | | actgtc-3′ |
| 30mers (s) | (SEQ ID NO: 8): | 5′-ctgaagccagttaccttcg |
| | | gaaaaagagtt-3′ |
| 30mer (as) | (SEQ ID NO: 9) | 5′-cagagcgcagataccaaat |
| | | actgtccttct-3′ |

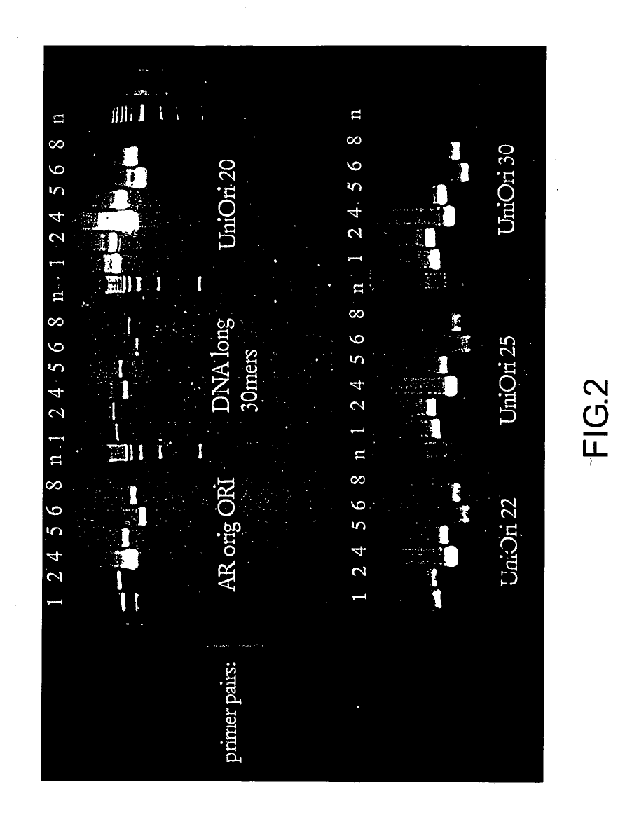

As shown in FIG. 2, the origin of replication designed primers provides excellent primer sites for amplifying the entire sequence of the six illustrative plasmids pEGFP, (lane 1), pEGFP (lane 2), pSP64+DHFR (lane 4), pBSK +MT4 (lane 5), pTZ18 (lane 6) and pTZ18 +IL2 (lane 8). This is especially true for all lengths of primers tested, all of which showed significant amplification product. This Example illustrates the utility of “universal primers” in accordance with embodiments of the present invention.

### Example 2

### Plasmid DNA can be Directly Amplified from an Overnight Culture

Colonies of cells harboring one of several extrachromosomal DNA elements were picked, re-suspended in water, and added directly to the amplification reaction buffers and universal primers of the present invention. Extrachromosomal DNA: pEGFP (lanes 1 and 2), pSP64+DHFR (lane 4), pBSK+MT4 (lane 5), pTZ18 (lane 6) and pTZ18+IL2 (lane 8 ) were transformed into chemically competent E. Coli cells and streaked on agar plates containing ampicillin, as is well known in the art. Colony picked cells were either added directly to the amplification reaction, were resuspended in 5 μl of water and 2.5 μl added to the amplification reaction, were resuspended in 10 μl of water and 2.5 μl added to the amplification reaction, were resuspended in 15 μl of water and 2.5 μl added to the amplification reaction or were resuspended in 10 μl of water, heated for five minutes at 95° C., and 2.5 μl added to the amplification reaction.

Amplification reactions were performed in 25 μl, having 30 mM bicine tris, 6 mM (NH₄)SO₄, 200 μM dNTPs, 2.5 mM Mg²⁺, enhancers (combination of DMSO, sorbitol, and trehalose), 2.5 units Triplemaster Enzyme, at a pH of 8.9. Amplification cycling conditions were: (1) 93° C. for two minutes, (2) 94° C. for twenty seconds, (3) 68° C. for three and (4) one-half minutes, repeated for 24 cycles.

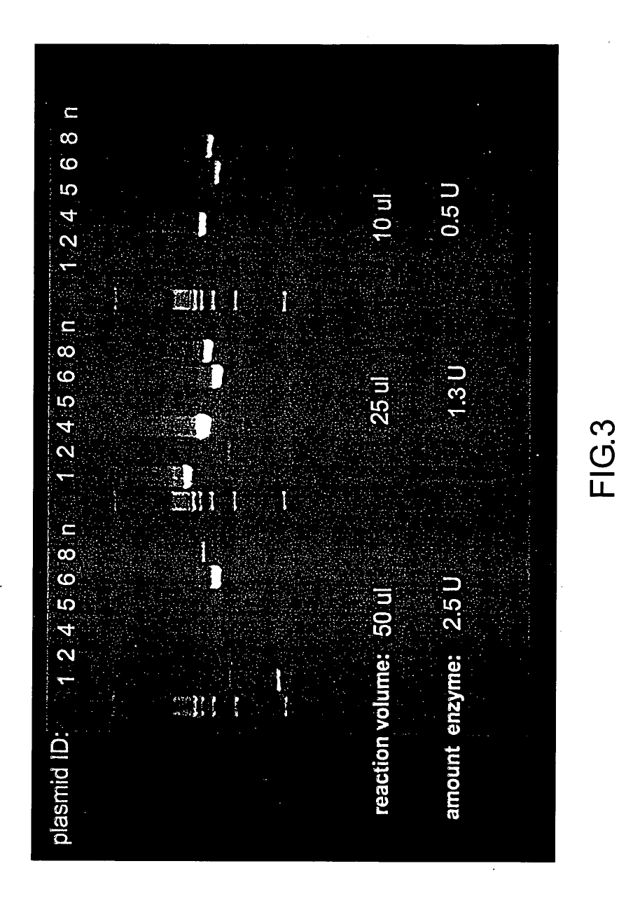

As shown in FIG. 3, resuspended picked colony DNA provided an excellent template for the amplification reaction buffers, primers and conditions of the present invention. Colonies re-suspended in five microliters provided large amounts of amplified extrachromosomal DNA. Heating the cells before use in the amplification reaction provided a small, if any, improvement. Additionally, numerous episomal genetic elements were amplified using the universal primers of the present invention and using the defined two-step PCR cycling parameters, as compared to most three-step PCR cycling parameters.

This example illustrates the utility of the invention for amplifying target plasmids directly from colony picked cells. In each case, a large amount of plasmid DNA was amplified in a fairly short amount of time using one embodiment of the modified PCR reaction buffer of the invention. Further, the universal primers of the invention provide the specificity and sensitivity to allow for two-cycle PCR reactions, thereby shortening the time required to perform a PCR on a target template.

### Example 3

### Plasmid DNA can be Directly Amplified from Purified Plasmid DNA, Glycerol Stocks or Colony Picked Cells

Target extrachromosomal DNA from either purified plasmid DNA (purified using Eppendorf Maxiprep, product # 955150066), a glycerol stock, or cultured cells were amplified using the methods and compositions of the present invention. Amplification buffer and primer (UniOri30, Table 7) were as described in Examples 1 and 2, which included 1.25 units of Eppendorf Triple Master polymerase. Template DNA used in the reaction was either 2.5 ng of purified extrachromosomal DNA, 2.5 μl of glycerol stock or 2.5 μl of a picked colony from an agar plate resuspended in 15 μl water. Note also that four different universal primer concentrations were used for each element source (purified, glycerol and culture) within the amplification reactions, a 0.3 μM, 0.15 μM, 0.075 μM and 0.6 μM. Episomal genetic elements from each source included: lane 1—pEGFP, lane 2—pTTQ, lane 4—pSP64+DHFR, lane 5—pBSK+MT4, lane 6—pTZ18 and lane 8—pTZ18+IL2. Product or amplicons from each resultant amplification reaction were visualized by running ⅓ of each reaction on a 1% ethidium bromide stained agarose gel.

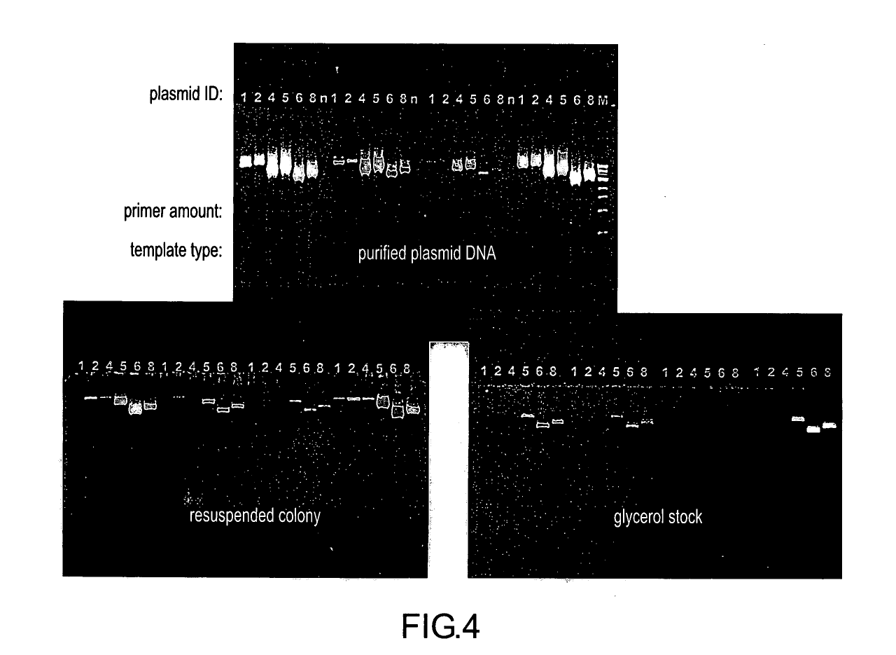

As shown in FIG. 4, the buffers and primers of the present invention show utility in supporting long range PCR for several different ColE1 based plasmids and from several different sources (purified, glycerol stock and picked culture). In addition, several different universal primer concentrations supported excellent amplification of full-length episomal genetic elements.

The Example illustrates the utility of directly amplifying target DNA from glycerol stocks or picked cultured cells resuspended in water. This data also shows that the universal primers of the present invention prime amplification reactions from numerous episomal genetic elements.

### Example 4

### Amplified Episomal Genetic Element DNA Has Excellent Sequencing Quality

A series of transformed chemically competent E. Coli host cells were harvested for direct colony amplification of the target extrachromosomal DNA. Target elements were either directly amplified from the colony picked cells using the methods and compositions of the present invention (See below) or were first purified using the Qiagen miniprep kit. Amplified target DNA were next sequenced using ABI Big-Dye Chemistry and associated equipment, and scored using the PHRED standard. Note that DNA prepared via growth and purification procedures were prepared using the manufacturer recommended steps (Qiagen controls).

Amplification reaction conditions were performed in 30 mM bicine, 200 mM tris, 6 mM ammonium sulfate, 2.5 mM magnesium ions, 100 μM dNTPs, 0.3 μM UniOri30 universal primer pair, enhancer set (trehalose, sorbitol, DMSO), 2.5 units Triple Master Enzyme, pH 8.85 to 8.90. Volumes of amplified DNA was sequenced using M13 forward sequencing primer with quarter reactions.

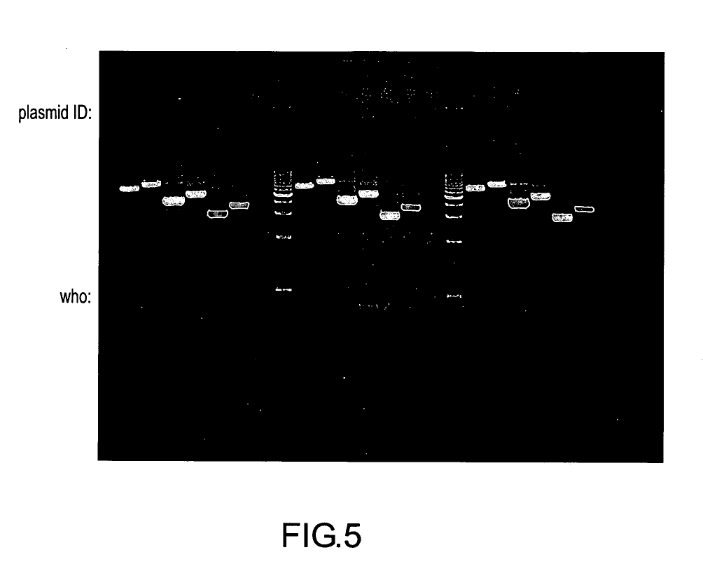

As shown in FIG. 5 and Table 8, visualization of illustrative plasmid templates and sequencing results indicate that methods and compositions of the present invention provide excellent amplification yield at a high quality. This data also indicates that the in vitro amplified DNA, using the universal primers of the present invention, can be used directly in sequencing reactions without the need for an additional post-amplification clean-up step. However, it was noted that as the amount of template volume was decreased the sequencing quality increased, suggesting that excess dNTPs and universal primer were likely interfering with the sequencing reaction. Taken together, the present invention provides universal primers that can be used to amplify, with high fidelity, pUC19, pGEM, and pTZ+IL2.

TABLE 8
Sequence Analysis

| | Total Volume of Amplified | |
|---|---|---|
| | Product Used in Sequencing | Number of |
| Sample ID | Reaction | Quality Scores ⊃ 20 |
| pUC19 | 4 μl | 114, 195, 352 |
| pUC19 | 2 μl | 325, 289, 167 |
| pUC19 | 1 μl | 550, 526, 215 |
| pUC19 | 0.5 μl | 693, 591 |
| pUC 19 - Qiagen | 4 μl | 662, 738 |
| Purified Prior to | | |
| Sequencing | | |
| pUC19 - Qiagen | 2 μl | 762, 748 |
| Purified Pior to | | |
| Sequencing | | |
| pUC19 Control | | 756 |
| PGEM | 4 μl | 131, 151, 64 |
| PGEM | 2 μl | 269, 374, 229 |
| PGEM | 1 μl | 514, 590, 507 |
| PGEM | 0.5 μl | 588, 650 |
| pGEM - Qiagen | 4 μl | 693, 608 |
| Purified Prior to | | |
| Sequencing | | |
| pGEM - Qiagen | 2 μl | 715, 669 |
| Purified Prior to | | |
| Sequencing | | |
| pGEM Control | | 743 |
| pTZ + IL2 | 4 μl | 115, 194, 127 |
| pTZ + IL2 | 2 μl | 623, 590, 330 |
| pTZ + IL2 | 1 μl | 693, 549, 443 |
| pTZ + IL2 | 0.5 μl | 522, 714 |
| pTZ + IL2 - Qiagen | 4 μl | 723, 665 |
| Purified Prior to | | |
| Sequencing | | |
| pTZ + IL2 - Qiagen | 2 μl | 736, 741 |
| Purified Prior to | | |
| Sequencing | | |
| pTZ + IL2 Control | | 741 |

Data from FIG. 5 and Table 8 indicate that robust amplification yields provide an excess of sequencing template for sequencing the episomal genetic element, such that volumes of starting material should be either cleaned-up using Qiagen kits (to remove excess dNTPs or primer) or other like cleaning steps, or be diluted down to dilute out the excess dNTPs and primer. To confirm that dilution of the amplification sample would provide adequate quality plasmid for sequencing, and to confirm that the amplification reaction provides full length episomal genetic elements, a second series of experiments was performed, using the same amplification buffer and universal primer set, but modified amounts of primer pairs in the amplification reaction and volumes for sequencing.

As shown in Table 9, smaller levels of primer in the amplification reaction, and smaller volumes of amplified product from colony picked cells, supported high quality sequencing and again supported the finding that the methods and compositions of the present invention provided for the amplification of full length episomal genetic elements. Note that when sequenced portions of pGEM were compared to a standard pGEM sequence, the fidelity of the present methods and compositions was excellent (note that 98.5% accuracy is considered excellent quality as determined by ABI sequencing guidelines).

TABLE 9
Variable Primer Concentrations and Dilution Volumes

| | | | | % Accuracy |
|---|---|---|---|---|
| | Primer | | Number of | (Sequence |
| | Con- | Sample | Quality | Autoscore, |
| Template Target | centration | Volume | Scores ≧20 | ABI, Inc.) |
| IL2 gene in | 0.15 μM | 0.1 μl | 686, 724, 701 | — |
| pTZ18 backbone | | | | |
| PGEM | 0.15 μM | 0.1 μl | 689, 715, 694 | 98, 97.3, 97.8 |
| pUC19 | 0.15 μM | 0.1 μl | 683, 661, 728 | — |
| IL2 gene in pTZ | 0.3 μM | 0.1 μl | 699, 738, 720 | — |
| backbone | | | | |
| PGEM | 0.3 μM | 0.1 μl | 731, 722, 756 | 99.1, 98.2, |
| | | | | 99.8 |
| pUC19 | 0.3 μM | 0.1 μl | 665, 717, 523 | — |
| IL2 gene in pTZ | 0.15 μM | 0.2 μl | 718, 707, 728 | |
| backbone | | | | |
| PGEM | 0.15 μM | 0.2 μl | 656, 688, 712 | 96.9, 97.1, |
| | | | | 98.2 |
| pUC19 | 0.15 μM | 0.2 μl | 667, 664, 708 | |
| IL2 gene in pTZ | 0.3 μM | 0.2 μl | 728, 699, 748 | |
| backbone | | | | |
| PGEM | 0.3 μM | 0.2 μl | 728, 759, 726 | 99.1, 98.4, |
| | | | | 99.1 |
| pUC19 | 0.3 μM | 0.2 μl | 716, 651, 720 | |
| IL2 gene in pTZ | 0.15 μM | 0.5 μl | 682, 712 | |
| backbone | | | | |
| PGEM | 0.15 μM | 0.5 μl | 665, 664 | 98.2, 98.2 |
| pUC19 | 0.15 μM | 0.5 μl | 636, 665 | |
| IL2 gene in pTZ | 0.3 μM | 0.5 μl | 567, 709 | |
| backbone | | | | |
| PGEM | 0.3 μM | 0.5 μl | 623, 559 | 99.1, 97.5 |
| pUC19 | 0.3 μM | 0.5 μl | 718, 537 | |

The preceding results illustrate the utility of the invention as a replacement or companion for preparation of high quality episomal or extrachromosomal DNA via conventional growth and purification techniques. Both PHRED and accuracy scores illustrate that methods and compositions of the present invention can be used to directly amplify full length episomal genetic element DNA. The portions of sequenced pGEM showed that the amplified DNA is of high quality, equivalent to miniprep prepared DNA.

### Example 5

### In Vitro Amplified Closed Circle Plasmid DNA

A reaction buffer was optimized for in vitro amplification of plasmid DNA directly from a colony, and for further ligating the amplified plasmid DNA back into closed circular DNA within the same reaction. The amplification and ligation reaction occurs in the same reaction buffer and is therefore considered one-step.

An amplification buffer having 7.5 mM magnesium acetate, 10 mM ammonium chloride, 1 mM DTT, 1 mM β-NAD, 500 μM dNTPs, 0.4 μM of a sense and anti-sense universal primer (See Table 5), 2 units Triple Master Enzyme mix, and where appropriate, 0.019 μg Tsc DNA ligase (Prokarya) (for +ligase reactions) were mixed into a “1×” buffer. Re-suspended colonies were mixed with amplification buffer (+ or −ligase) to either amplify linear plasmid DNA or to amplify and ligate the plasmid DNA into closed circular DNA. Thermal cycling conditions included an initial denaturation step, 93° C. for twenty seconds, 28 cycles of 93° C. for twenty seconds steps, 58° C. for twenty seconds, and 68° C. for two minutes step.

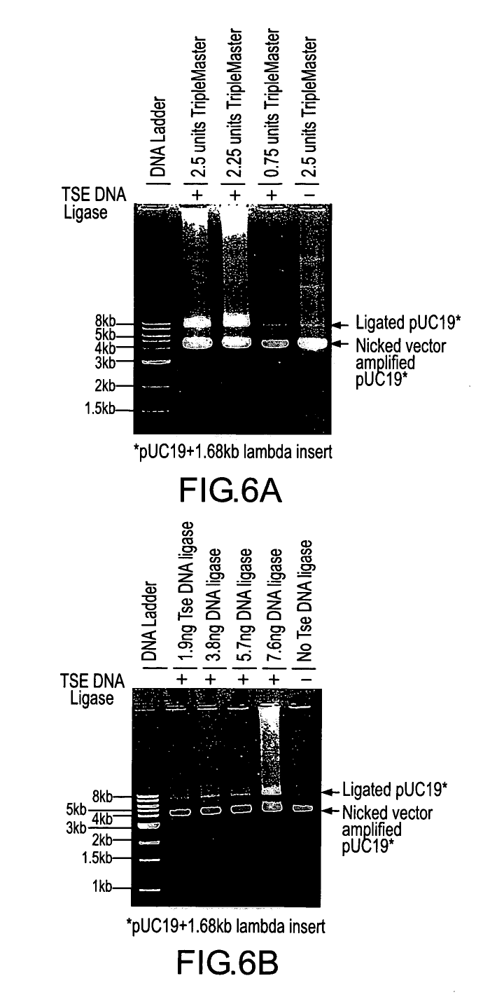

As shown in FIGS. 6A and B an impressive amount of in vitro amplified plasmid DNA is ligated into the circular species. FIG. 6A shows that ligated product was obtained when polymerase and ligase were added to the same one step reaction, and that increasing amounts of the ligated species were formed with increasing amounts of polymerase in the reaction. FIG. 6B illustrates that only a small increase in the ligated species occurred with increasing amounts This Example illustrates the utility of the present invention in providing a one-step amplification/ligation reaction directly for a colony pick.

### Example 6

### One-Step Amplification/Ligation Reaction Produces Circular Product

The following Example was performed to verify that a ligated circular form of plasmid was being produced in the one-step amplification/ligation reaction. Chemically competent E. Coli cells were transformed with a pUC 19 plasmid having a 1.68 Kb lambda insert at a single site. Colonies were picked and the one-step amplification/ligation reaction of the invention performed, briefly: each 2 microliter colony pick was placed into a 50 μl reaction volume having 7.5 mM magnesium acetate, 10 mM ammonium chloride, 1 mM DTT, 1 mM beta-NAD, 500 μM dNTPs, 0.4 μM reverse and forward universal primers (See Table 5), and 2 Units of Triple Master polymerase mix (Eppendorf, product # 954140229). Samples that include ligase also included 0.019 μg Tsc DNA ligase (Prokarya). Thermal cycling conditions included an initial denaturation step, 93° C. for twenty seconds, 28 cycles of 93° C. for twenty seconds steps, 58° C. for twenty seconds, and 68° C. for two minutes step.

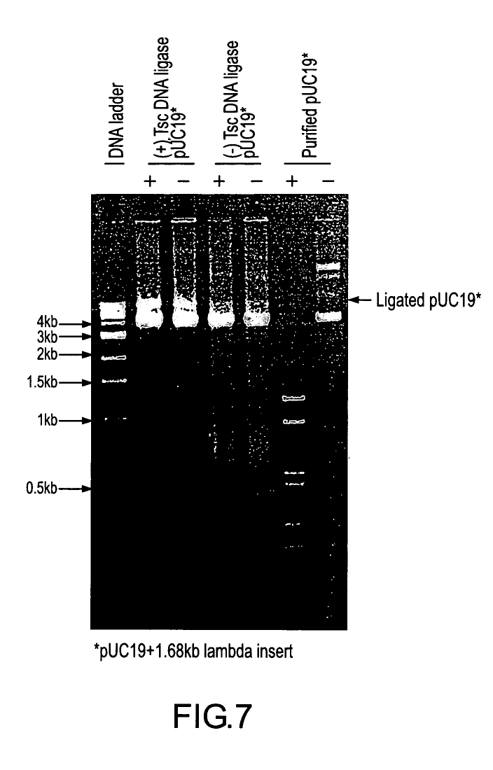

As shown in FIG. 7, the above reaction conditions resulted in the production of closed circular product. This again illustrates the utility of the present invention for a combined polymerase/ligase driven reaction buffer.

### Example 7

### Amplification Buffers of the Present Invention Buffer Mg²⁺

Amplification reactions were performed similar to as described in Examples 2 and 3, under varying concentrations of Mg²⁺ so as to illustrate that the compositions and methods of the present invention provide Mg²⁺ buffering capacity. As above, several different extrachromosomal DNA elements were amplified using the UniOri30 primer pair in the amplification reaction mixture described in Table 5. Cycling conditions were as above in Example 2, using the two-step PCR program.

Mg²⁺ concentrations were increased from 2.5 mM to 6 mM to illustrate that the bicine containing buffers of the present invention, are resistant to higher Mg²⁺ reaction concentrations (bicine is a known Mg²⁺ chelator).

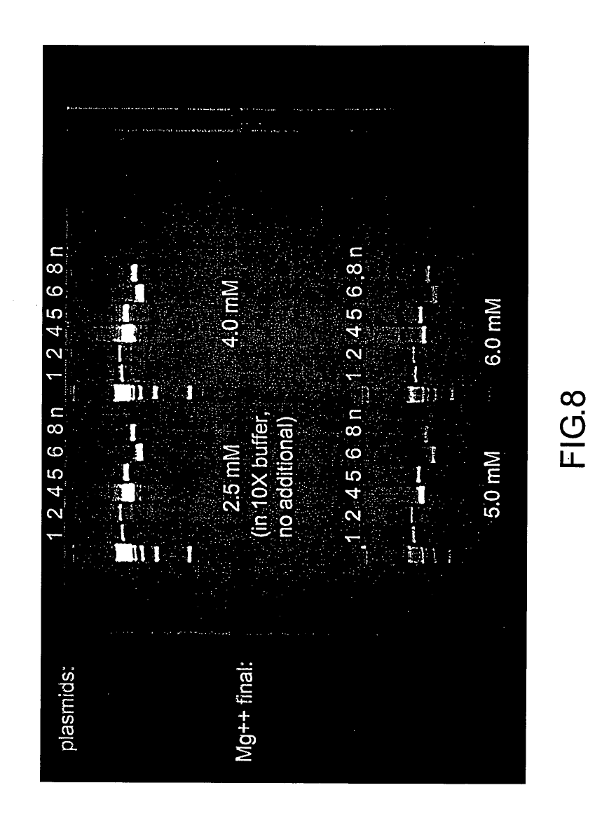

As shown in FIG. 8, the present amplification buffer supports a wide range of Mg²⁺ ion concentration during the amplification reaction while supporting high levels of polymerase activity. This result is seen for each of the different extrachromosomal DNA elements.

### Example 8

### UniOri30 Primer Pair Supports Two-Step PCR

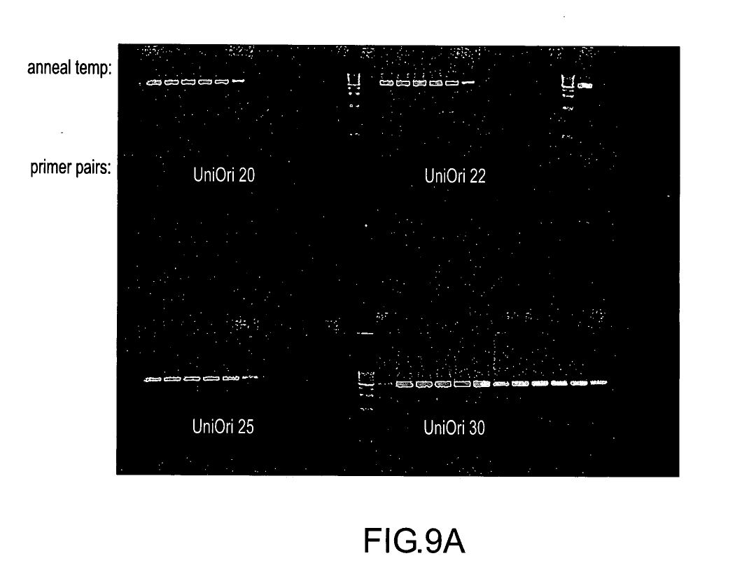

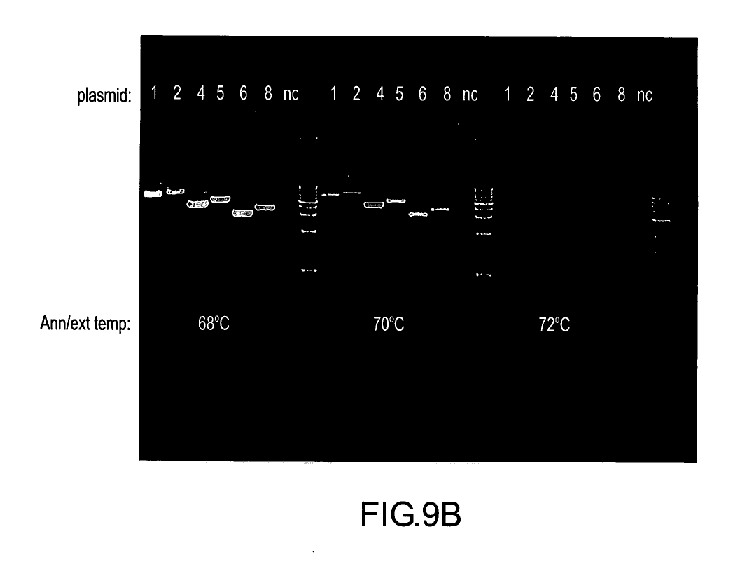

A series of amplification reactions were performed on pBSK+NT4 plasmid using either the UniOri20, UniOri22, UniOri25 or UniOri30 primer pair to determine if a common annealing and extension condition can be obtained. Reaction buffer conditions were as described in the Examples above, using 1 ng of template and 0.3 μM or each primer pair. Two units of Triple Master Polymerase was used in each reaction. Cycling conditions included 93° C. for two minutes, 94° C. for twenty seconds, and a varying temperature gradient for annealing that went from 55 to 72 degrees C. Extension steps were conducted for five minutes and thirty seconds. As shown in FIG. 9A, only the UniOri30 supported annealing above 65° C., a minimum temperature required for most thermophilic polymerase enzymes to extend off of a primed template. Importantly, UniOri30 actually annealed to template DNA even at temperatures within most enzyme polymerase activity, i.e., 68 to 72° C.

From the above data, it was surmised that a two-step PCR cycling condition was possible using the Triple master polymerase and UniOri30 at an annealing and extension temperature of 68° C. As shown in FIG. 9B, several plasmid targets were effectively amplified using the UniOri30 primer pair and a 93° C. for two minute, 94° C. for twenty seconds and a 68° C. for six minute cycle. Each reaction was performed either for 25 (left-hand ) or 30 (right-hand panel) cycles. The above example illustrates the utility of the present invention for amplifying episomal genetic elements using two-cycle PCR.

### Example 9

### Amplified DNA from Colony Picked Cells is Predominately Endotoxin-Free

Target extrachromosomal DNA from colony picked cells was amplified using the methods and compositions of the present invention. For example, amplification reactions were performed as substantially shown in Example 2. Endotoxin levels present in the amplified product were determined by test known in the art and compared to endotoxin levels present with substantially an equivalent amount of DNA isolated via the Qiagen ET-free Midiprep. Note that this Qiagen kit has been marketed/designed, at least partially, for the capacity for keeping endotoxin levels at a minimal levels.

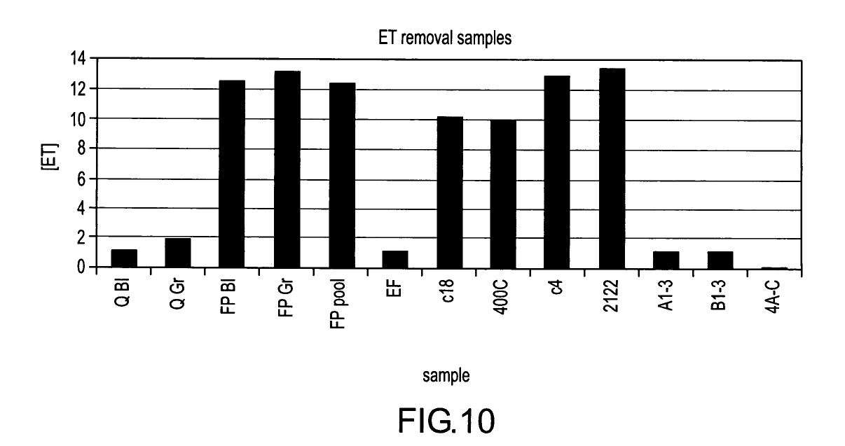

Data as shown in FIG. 10 illustrates that DNA amplified directly from cells using the methods and compositions of the present invention showed very low levels of endotoxin, comparable to the levels found in DNA isolated using the Qiagen ET-free method and compositions. More specifically, the first two bars as shown in FIG. 10, QB1 and Q Gr, are Qiagen minipreps, FP, B1, FP Gr and FP pool were prepared as in U.S. patent application Ser. No. 10/387,646 ('646). The bar labeled EF is endotoxin free Qiagen midi prep, and the subsequent four bars are spin devices used to attempt to remove endotoxin from samples as prepared in '646 application (C18 (50% composition like the PerfectPure Tip); 400C (50% Tosoh Butyl 400C); C4 (Macherey-Nagel C4 resin); and 2122 (100% gur2122 sintered particles)). The remaining three bars are samples as prepared using the methods and compositions in Example 2. Note that 4A-C was a sample that had a failure to show amplified DNA.

The data in this Example illustrates the utility of directly amplifying target DNA from colony picked cells, while maintaining a predominately endotoxin free environment, important because of endotoxin effects on cell cultures during transfections and other like procedures. As such, target DNA amplified using the methods and compositions of the present invention provide a superior amplification product for use in transfections, transformations, and other like procedures.

### Example 10

### Modified Universal Primers are Resistant to Polymerase Proofreading Activity

Compositions of the present invention were used to amplify target plasmid DNA using LNA modified universal primers. A universal primer pair was designed to hybridize within the origin of replication of the plasmid, and further each oligonucleotide of the pair was designed to include a locked nucleic acid at the n-1 position. LNA modified primers have previously been shown to provide significant protection against exonuclease activity associated with proofreading DNA polymerase enzymes (See Di Giusto et al., supra). A pair of non-modified universal primers was prepared for comparison with the modified universal primer pairs.

In vitro amplification reactions were performed as described above using the reaction conditions as shown in Table 10.

TABLE 10
LNA Modified Universal Primer Pair

| Reagent | Stock Concen. | 1X Concen. | 1X Volume | 110X mix |
|---|---|---|---|---|
| PCR Buffer | 10X | 1X | 1 μl | 110 μl |
| From Example | | | | |
| above (includes | | | | |
| 2.5 mM Mg) | | | | |
| DNTPs | 10 mM | 75 μM | 0.075 μl | 8.25 μl |
| UniOri30 or | 10 μM | 0.10 μM | 0.1 μl | 11 μl |
| LNA modified | | | | |
| UniOri30 | | | | |
| Triple Master | 5 U/μl | 0.25 units | 0.05 μl | 5.5 μl |
| Taq | | | | |
| H₂O | | | 7.775 μl | 855.25 μl |
| Template DNA | | | | |

Template:Dilution: 9 μl mix with 1 μl diluted template
Cycling Conditions: 6 step = (1) 94° C. for 2 min; (2) 94° C. for 20 sec; (3) 68° C. for 3 min 30 sec; (4) back to step (2) 29 times; (5) 4° C. hold; (6) End cycle.

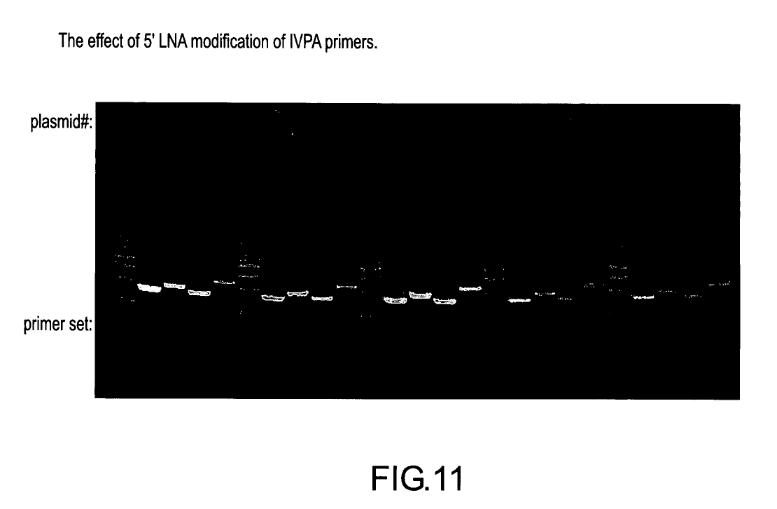

Data as shown in FIG. 11 illustrates that the LNA modified primers, see Table 10(a), were extendable and provided substantial protection against primer degradation (note plasmid number, identity and host cell—also in Table 10(a).

TABLE 10(a)
Identification of Plasmid Samples and Primer Modification for
LNA Primers

| | | |
|---|---|---|
| Plasmid # | Identity | Host Cells |

| | | |
|---|---|---|
| 1 | pUC 19 | DH10b |
| 2 | pBS2 | HB101 |
| 3 | pUC 18 | XL2 blue |
| 4 | pGEM | DH5a |

Modified primers in LNA/phosphorothioate experiment

| | | |
|---|---|---|
| Primer Set | Sense Primer | Antisense Primer |

| | | |
|---|---|---|
| A | CACtgattaagcattggtaactgtc | Aggcacctatctcagcgatctgtc |
| B | CACtgattaagcattggtaactgtc | AGgcacctatctcagcgatctgtc |
| C | CACtgattaagcattggtaactgtc | AGGcacctatctcagcgatctgtc |
| D | cactgattaagcattggtaactgtc | aggcacctatctcagcgatctgtc |
| E | c*a*c*t*gattaagcattggtaactgtc | a*g*g*c*acctatctcagcgatctgtc |

DNA bases in lower case; LNA bases in CAPS; Phosphothioate bases noted with *

### Example 11

### Amplification and Sequencing of Target Plasmid DNA

A series of different host cells, each transformed with a different plasmid, was harvested via direct colony pick. Colony picked cultures were diluted 1 to 10 in water and plasmid DNA amplified and sequenced using the methods and compositions of the present invention (See Table 11 for overview of host cell/plasmid type combinations, Table 12 for amplification reaction conditions and Table 13 for sequencing data).

Amplified target DNA was sequenced using ABI Big-Dye Chemistry with associated equipment, and scored using PHRED standard. Note that the ABI 3700 and ABI 3100 sequencing instruments were both used in the context of this example.

Data from this Example shows the superior results obtained by amplifying target plasmid DNA directly from a colony pick for subsequent sequencing. Embodiments of the present invention can be used to directly sequence target portions of plasmid or other extrachromosomal DNA harbored in a target cell directly from the host cell using the amplification reactions of the present invention.

TABLE 11
Plasmid Identification Panel

| | Observed | Sequence Primer Match | | | Host | | | Expected | |
|---|---|---|---|---|---|---|---|---|---|
| | Linear | M13forw | SP6 | T7 | Cells | | Good | Size Range- | |
| Plasmid | Amplicon | gtaaaacgac- | atttaggtg- | taatacgact- | For | MTD | Sequence | Supercoiled | |
| ID | Size (Kb) | gacggccagt | acactatag | cactataggg | GSS | List | Data | (Kb) | ng/μl |
| pEGFP- | 6.5 | no | | | DH10b | ** | xxx | 8-10 | 290 |
| tub | | | | | | | | | |
| PTTQ | 7.9 | yes | yes | no | | | ** | 10-12 | 69 |
| pSP64- | 4.1 | | yes | yes | | | * | 6-8 | 231 |
| DHFR | | | | | | | | | |
| pTZ18 | 2.9 | yes | no | yes | XL1 | | **** | 1.5-2.5 | 303 |
| | | | | | blue | | | | |
| pTZ + IL2 | 3.4 | yes | no | yes | Top 10 | | **** | 2-3 | |
| pUC19 | 2.7 | yes | | | XL1 | ** | **** | | |
| | | | | | blue | | | | |
| PGEM | 3.2 | yes | | | DH5a | | *** | | |
| pPICZ-E | 5 | | | | pichia | ** | \hat{ }\hat{ }\hat{ }\hat{ } | 3.4 | |
| pFastBac | 6 | yes | | | XL1 | ** | ** | 4.8 | |
| dual | | | | | blue | | | | |
| pQE Xa | 6.5 | | | | XL1 | ** | * | | |
| | | | | | blue | | | | |
| pCR- | 4 | yes | | yes | | ** | *** | | |
| TOPC | | | | | | | | | |
| PGEX | 4.2 | yes | | | 4 | ** | ** | | |
| pUC19 | | | | | XL1 | | | | |
| | | | | | blue | | | | |
| pET19 | | | | | DH10b | | | | |

Although each of the above described plasmid preparations (See Table 11) was tested using the methods and compositions as described in the present Example, only data generated using plasmids pFastBac, pQE Xa, pCR-TOPC, and pGEX are shown below in Table 13. Note that in vitro amplification reaction conditions have been summarized in Table 12.

TABLE 12
Reaction Mixture For Plasmid Amplification

| | | | | | | 100rxn | 500rxn | 2500rxn |
|---|---|---|---|---|---|---|---|---|
| | | | | | | kit: | kit | kit |
| | | | | | | 111X | 555X | 2750X |
| | Stock | 1X | | 10X | 100X | (11% | (11% | (10% |
| Reagent | Conc. | Conc. | 1X Vol. | Mmix | Mmix | Over) | Over) | Over) |
| PCR | 10X | 1X | 1 μl | 10 μl | 100 μl | 111.0 μl | 555.0 μl | 2750 μl |
| Buffer | | | | | | | | |
| Tuning* | | | | | | | | |
| dNTPs | 10 mM | 75 μM | 0.075 μl | 0.75 μl | 7.5 μl | 8.33 μl | 41.63 μl | 206.3 μl |
| primer pair | 5 μM | 0.1 μM | 0.2 μl | 2 μl | 20 μl | 22.2 μl | 111.0 μl | 550 μl |
| (UniOri30) | | | | | | | | |
| template | 1 ng/μl | 1 ng | 1 μl | | | | | |
| DNA (See | | | | | | | | |
| Table _{——}) | | | | | | | | |
| Triple | 5 U/μl | 0.25 units | 0.05 μl | 0.5 μl | 5 μl | 5.55 μl | 27.75 μl | 137.5 μl |
| Master | | | | | | | | |
| Taq | | | | | | | | |
| H₂O | | | 7.675 μl | 76.8 μl | 767.5 μl | 851.9 μl | 4259.6 μl | 21.106 ml |

*Buffer constituents include final concentration of 30 mM bicine, 200 mM tris, 6 mM ammonium sulfate, and 2.5 mM Mg²⁺.

As shown in Table 13, plasmid DNA harbored by numerous different host cells can be amplified using the universal primers of the present invention and accurately sequenced in accordance with embodiments of the present invention. Note also that the same amplification buffer was used regardless of host cell or plasmid identification using the universal primers of the present invention.

TABLE 13
Sequencing Analysis

| | | Primer | | |
|---|---|---|---|---|
| | Source and | Conc. | Amplicon Vol. | PHRED Q20 |
| plasmid ID | Dilution | (μM) | (μl) | Score |
| PFastBac | Colony Pick 1:10 | 0.15 | 0.5 | 450 |
| | dilution in water | | | |
| PFastBac | Colony Pick 1:10 | 0.15 | 1 | 404 |
| | dilution in water | | | |
| PFastBac | Colony Pick 1:10 | 0.15 | 0.75 | 425 |
| | dilution in water | | | |
| PFastBac | Colony Pick 1:10 | 0.15 | 0.75 | 467 |
| | dilution in water | | | |
| PFastBac | Colony Pick 1:10 | 0.15 | 1 | 476 |
| | dilution in water | | | |
| pQE Xa | Colony Pick 1:10 | 0.15 | 0.75 | 425 |
| | dilution in water | | | |
| pQE Xa | Colony Pick 1:10 | 0.15 | 0.75 | 415 |
| | dilution in water | | | |
| pQE Xa | Colony Pick 1:10 | 0.15 | 1 | 445 |
| | dilution in water | | | |
| pQE Xa | Colony Pick 1:10 | 0.15 | 1 | 429 |
| | dilution in water | | | |
| pCR-TOPC | Colony Pick 1:10 | 0.15 | 1 | 655 |
| | dilution in water | | | |
| pCR-TOPC | Colony Pick 1:10 | 0.15 | 1 | 644 |
| | dilution in water | | | |
| pCR-TOPC | Colony Pick 1:10 | 0.15 | 1 | 528 |
| | dilution in water | | | |
| pCR-TOPC | Colony Pick 1:10 | 0.15 | 2 | 591 |
| | dilution in water | | | |
| pCR-TOPC | Colony Pick 1:10 | 0.15 | 1 | 419 |
| | dilution in water | | | |
| pCR-TOPC | Colony Pick 1:10 | 0.15 | 1 | 542 |
| | dilution in water | | | |
| pGEX | Colony Pick 1:10 | 0.15 | 0.5 | 498 |
| | dilution in water | | | |
| pGEX | Colony Pick 1:10 | 0.15 | 1 | 495 |
| | dilution in water | | | |

The present Example illustrates that the universal primer and amplification buffer of the present invention can be used on a series of different host cell/plasmid type combinations to provide amplified and sequence ready plasmid DNA. Sequencing reactions can be performed directly on amplified plasmid DNA, where high quality of DNA (PHRED scores above 450 are indicative of appropriate sequencing accuracy) is anticipated and shown for the present experiment.

The invention has been described with reference to specific examples. These examples are not meant to limit the invention in any way. It is understood for purposes of this disclosure, that various changes and modifications may be made to the invention that are well within the scope of the invention. Numerous other changes may be made which will readily suggest themselves to those skilled in the art and which are encompassed in the spirit of the invention disclosed herein and as defined in the appended claims.

This specification contains numerous citations to patents, patent applications, and publications, each is hereby incorporated by reference for all purposes.

1. A method for amplifying a circular nucleic acid template, comprising:
contacting the template with a reaction mixture comprising a thermostable polymerase, individual nucleotides, and forward and reverse primers complementary to a common region within the template; and
thermally cycling the template in the reaction mixture through a plurality of cycles comprising a denaturation phase and an extension phase to produce multiple amplicons of said template;
wherein the 5′ end of said forward primer is proximal to the 5′ end of said reverse primer and distal to the 3′ end of said reverse primer within said common region.

2. The method according to claim 1, wherein said circular nucleic acid template is an extrachromosomal nucleic acid, and said common region is a conserved region within said extrachromosomal nucleic acid.

3. The method according to claim 2, wherein said extrachromosomal nucleic acid is a plasmid, and said conserved region is an origin of replication.

4. The method according to claim 3, wherein said conserved region is a portion of a ColE1-based plasmid origin of replication.

5. The method according to claim 2, wherein said conserved region is a portion of a gene encoding for drug resistance.

6. The method according to claim 1, wherein said common region is from about 80 to 150 base pairs in length.

7. The method according to claim 1, wherein the 5′ ends of said primers hybridize to opposite strands of said template about 10 to 50 base pairs apart.

8. The method according to claim 1, wherein the 5′ ends of said primers are adjacent each other on opposite strands of said template.

9. The method according to claim 1, wherein the 5′ ends of said primers overlap each other by about 1 to 10 base pairs on opposite strands of said template.

10. The method according to claim 5, wherein said gene is an ampicillin gene.

11. The method according to claim 1, wherein said reaction mixture further comprises a reaction buffer having a pH of between 7.8 and 9.0 and comprising a weak organic acid and a weak organic base.

12. A method for reducing endotoxin levels in extrachromosomal nucleic acid derived from cultured cells, said method consisting essentially of:
harvesting at least one colony of cells comprising said extracellular nucleic acid;
resuspending said cells in an aqueous medium; and
amplifying said extracellular nucleic acid using a method according to any one of claims 1-5; wherein said amplicons are substantially free of endotoxin.

13. The method according to claim 12, comprising the additional step of heating said cells in said aqueous medium prior to said amplification step.

14. The method according to claim 12, wherein from one to five cell colonies are harvested in said harvesting step.

15. A composition for amplifying a circular nucleic acid template having a conserved region, said composition comprising forward and reverse primers from about 10 to 40 base pairs in length, wherein said primers are complementary to opposite strands of said template within said conserved region and wherein the 5′-end of the forward primer hybridizes to the sense strand of the template in close proximity to the reverse primer when hybridized to the antisense strand of the template.

16. A composition according to claim 15, wherein the 5′ end of said forward primer is proximal to the 5′ end of said reverse primer and distal to the 3′ end of said reverse primer around the circumference of said template when said primers hybridize to opposite strands of said template.

17. A composition according to claim 15, wherein said conserved region is selected from among a bacterial origin of replication, a yeast two micron origin, a lambda cos site, or a drug resistance gene.

18. A composition according to claim 15, comprising a primer pair selected from Table 7.

19. A reaction buffer for amplifying a nucleic acid template, comprising a weak organic acid and a weak organic base and having a pH of between about 7.8 and 9.0.

20. The reaction buffer according to claim 19, wherein the weak organic acid is selected from the group consisting of bicine, tricine, TAPSO, CAPSO, EPPS, Hepes, CHES, Taurin, MOPS, AMPSO, and mixtures thereof.

21. The reaction buffer according to claim 19, wherein the weak organic base is selected from the group consisting of tris, bis-tris, imidazole, and tris-propane.

22. The reaction buffer according to claim 19, further comprising a salt at a concentration of from about 30 mM to about 300 mM salt.

23. The reaction buffer according to claim 22, wherein the salt has a concentration of from about 30 mM to about 100 mM.

24. The reaction buffer according to claim 19, further comprising a magnesium containing compound.

25. The reaction buffer according to claim 19, wherein the pH of the buffer is between about 8.8 and about 8.9.

26. A method for amplifying a nucleic acid template, comprising:
contacting the template with a reaction mixture comprising a thermostable polymerase, individual nucleotides, and at least one primer in a reaction buffer according to any one of claims 19-25; and
thermally cycling the template in the reaction mixture through a plurality of cycles comprising a denaturation phase and an extension phase to produce multiple amplicons of said template.

27. The method according to claim 26, wherein said nucleic acid template is a circular nucleic acid template and said reaction mixture comprises forward and reverse primers having 5′ ends that hybridize to adjacent base pairs on said circular nucleic acid template.

28. The method according to claim 27, wherein said circular nucleic acid template is an extrachromosomal nucleic acid, and said common region is a conserved region within said extrachromosomal nucleic acid.

29. A kit for amplifying extrachromosomal nucleic acid, said kit comprising a primer composition according to any one of claims 15-18.

30. A kit for amplifying extrachromosomal nucleic acid, said kit comprising a reaction buffer according to any one of claims 19-25.

31. The kit according to claim 29 or 30, wherein said forward and reverse primers have a locked-nucleic acid at position n-1 within each primer.
